(19) United States
(12) Patent Application Publication
(10) Pub. No.: US 2015/0030609 A1
(43) Pub. Date: Jan. 29, 2015

(54) DIAGNOSIS AND TREATMENT OF TRAUMATIC BRAIN INJURY

(71) Applicants: Subhra Mohapatra, Lutz, FL (US); Shyam S. Mohapatra, Lutz, FL (US); Keith Ronald Pennypacker, Wesley Chapel, FL (US); Mahasweta Das, Wesley Chapel, FL (US); Christopher Charles Leonardo, Apollo Beach, FL (US)

(72) Inventors: Subhra Mohapatra, Lutz, FL (US); Shyam S. Mohapatra, Lutz, FL (US); Keith Ronald Pennypacker, Wesley Chapel, FL (US); Mahasweta Das, Wesley Chapel, FL (US); Christopher Charles Leonardo, Apollo Beach, FL (US)

(73) Assignee: UNIVERSITY OF SOUTH FLORIDA, Tampa, FL (US)

(21) Appl. No.: 14/006,557

(22) PCT Filed: May 3, 2012
(86) PCT No.: PCT/US12/36335

§ 371 Date: Aug. 18, 2014

Related U.S. Application Data
(60) Provisional application No. 61/481,997, filed on May 3, 2011.

(51) Int. Cl. G01N 33/68; C07K 16/24

(52) U.S. Cl. CPC G01N 33/6863; C07K 16/24; G01N 2333/521; G01N 2800/28
USPC 424/158.1; 435/7.92; 435/7.1; 530/389.2

(57) ABSTRACT

The subject invention identifies CC chemokine ligand 20 (CCL20) as a novel biomarker for diagnosis of traumatic brain injury and/or neurodegeneration in the brain. The subject invention also provides treatment methods for traumatic brain injury and/or neurodegeneration in the brain by modulating systemic and/or brain-specific CCL20-CCR6 signaling. Also provided are uses of CCL20-CCR6 signaling a target for screening for therapeutic agents that are useful for treatment of traumatic brain injury.

### CROSS-REFERENCE TO RELATED APPLICATION

This application claims the benefit of U.S. provisional application Ser. No. 61/481,997, filed May 3, 2011, which is herein incorporated by reference in its entirety.

### BACKGROUND OF THE INVENTION

Traumatic brain injury (TBI), which afflicts an estimated 2.5 to 3.7 million Americans each year, is the leading cause of death in the United States. The risk of TBI is particularly significant for military service members in combat operations. It is estimated that 150,000-300,000 soldiers in the Operation Iraqi Freedom and Operation Enduring Freedom suffer from some level of traumatic brain injury (TBI) [1-4].

TBI is a complex pathological process that involves three overlapping phases: primary injury to brain tissue and cerebral vasculature caused by the initial impact to the head, secondary injury including neuroinflammatory processes triggered by the primary insult, and regenerative responses including enhanced proliferation of neural progenitor cells and endothelial cells.

The secondary injury, which results largely from the primary injury to the cerebral vasculature, is a progressive process that develops within a few hours to days after the primary injury. Specifically, primary injury to organs in trauma patients results in elevated circulatory levels of pro-inflammatory cytokines, cell mediators, and leukocytes including neutrophils, monocytes and lymphocytes. These pro-inflammatory mediators infiltrate into the brain parenchyma through a compromised blood brain barrier (BBB) caused by the primary injury [5-7]. Further, microglia, the resident macrophages of the brain, also release various pro-inflammatory factors after the primary injury and exert deleterious effects on neural cell survival.

Despite the daunting prevalence, the pathogenesis of TBI-induced brain injury is poorly understood. In addition, while the up-regulation of inflammatory pathways has provided some clues regarding the source and progression of TBI pathology, biomarkers useful for diagnosis and treatment of TBI have not been identified. Thus, there is a critical need for elucidating the pathogenesis and progression of TBI-induced injury. There is also a need for the identification of novel biomarkers useful for diagnosis and treatment of TBI. As will be clear from the disclosure that follows, these and other benefits are provided by the subject invention.

### BRIEF SUMMARY

The subject invention pertains to the use of CC chemokine ligand 20 (CCL20, also known as MIP-3α), as a novel biomarker for early detection of traumatic brain injury (TBI) and/or neurodegeneration in the brain. The subject invention also provides treatment methods for traumatic brain injury by modulating systemic and/or brain-specific CCL20-CCR6 signaling. Also provided are uses of CCL20-CCR6 signaling as a target for screening for therapeutic agents that are useful for treatment of TBI and/or neurodegeneration in the brain.

The subject invention is based at least in part on the surprising discovery that CCL20 is significantly up-regulated in the spleen and brain tissue after the primary TBI. After the induction of TBI in rats by the lateral fluid percussion (LFP) procedure, comprehensive gene expression analyses were performed. It was discovered that CCL20 expression was elevated in spleen tissue 12-24 hours post-TBI. Analysis of brain sections also showed that CCL20 immunoreactivity is abundant throughout the CA1 and CA3 pyramidal cell layers 48 hours post-TBI. Moreover, CCL20 expression was localized to the same cell layers that exhibited neurodegeneration (stained intensely for Fluoro-Jade) at 24 hours. CCL20 is also toxic to cultured oligodendrocytes.

In one embodiment, the subject invention provides a method for diagnosing TBI and/or neurodegeneration in the brain, comprising determining CCL20 level in a biological sample from a subject. The subject's CCL20 level may then be characterized in relation to TBI and the secondary injury caused by the TBI. Specifically, the degree of elevation in CCL20 level in the subject's biological sample, when compared to a predetermined reference value, indicates the presence of TBI as well as the severity of the secondary injury (e.g., neuroinflammation and/or neurodegeneration in brain tissue) caused by TBI.

In addition, the subject invention provides methods for treatment of TBI and/or neurodegeneration in the brain. In one embodiment, the method comprises modulating pro-inflammatory CCL20-CCR6 signaling in a subject who has TBI and/or neurodegeneration in the brain. In a further embodiment, the method for treating traumatic brain injury and/or neurodegeneration in the brain comprises the administration of an anti-inflammatory and/or a neuroprotective agent.

### BRIEF DESCRIPTION OF THE SEQUENCES

SEQ ID NO:1 is an amino acid sequence of human CC chemokine ligand 20 isoform 1 (GenBank Accession No. NP_{—}004582.1).

SEQ ID NO:2 is an amino acid sequence of human CC chemokine ligand 20 isoform 2 GenBank Accession No. NP_{—}0011235 18.1).

### BRIEF DESCRIPTION OF THE DRAWINGS

FIGS. 1A-C show that traumatic brain injury (TBI) induces neurodegeneration in different areas of the rat brain. Fluoro Jade (FJ) staining was performed on cryosections from rat brains to identify the damaged neurons 24 hours or 48 hours after the induction of mild lateral fluid percussion impact (LFPI). (A) Representative low magnification (40×) photomicrographs show FJ-positive neurons, which indicate neurodegeneration in cortex, hippocampus and thalamus 24 hours and 48 hours after LFPI. No degenerating neurons were observed in the corresponding brain regions of the sham animals. High magnification (400×) images from selected areas of respective sections are shown in the inset. Scale bars=500μ. (B) The FJ-positive neurons were quantitated using the Image J program. The histograms show the estimation of FJ-positive neurons in cortex, hippocampus and thalamus. Cortex showed the highest number of injured neurons compared to other regions. Higher numbers of FJ-positive neurons were observed after 24 hours of LFPI injury in all three regions. The numbers of FJ-positive neurons decrease 48 hours after TBI, but were significantly higher compared to sham animals. ** p<0.001 compared to sham animals. (C) Fluoro-Jade-labeled degenerating neurons were present throughout the brain, but were most prominent in hippocampus (a-c), cerebral cortex (d-f) and thalamus (g-i). In contrast to cortical and thalamic staining, which was diffusely distributed throughout these regions, hippocampal degeneration was consistently localized to the CA1 (a) and CA3 (b) pyramidal cell layers and was less prominent in the dentate gyrus (c). Scale bars=500 μm.

FIG. 2 shows that TBI causes DNA damage 24 hours after impact. (A, B) Photomicrographs of representative sections from rat cortex (A) or hippocampus (B) showing TUNEL histochemistry 24 hours after mild LFPI. TUNEL-positive nuclei (green fluorescence) were distributed throughout the ipsilateral cortex or hippocampus 24 h after TB. Intense signals were observed as rims on the nuclear boundaries with a diffuse homogeneous signal on the interior of the nucleus. Arrows indicate the TUNEL positive nuclei. (Scale bar 500μ). (C) Histograms show the number of TUNEL-positive nuclei in the cortex or hippocampus 24 hours or 48 hours after TBI. Significant increase in the TUNEL-positive nuclei at the 24 h time point indicates that the DNA damage occurs in these brain regions as early as 24 h post-TBI. At 48 hours after TBI the DNA damage was not significantly different in TBI animals compared to sham-treated animals. (** p<0.001 compared to sham animals).

FIG. 3 shows that mild TBI activates microglia 24 hours after impact. Some of these cells were CD11b-positive. This labelling was absent in the sham animals and was significantly less on the contralateral side or 48 h after TBI. (A) Confocal microscopic images show IB4-positive (Alexafluor 488-conjugated, green fluorescence), CD11b-positive (red fluorescence) or IB4/CD11b-positive (red-green overlap) microglia in representative sections of ipsilateral dentate gyrus 24 hours after mild TBI. The left column shows CD11b immunostaining, the middle column shows IB4 labelling, and the right column is an overlay of CD11b and IB4 double labelling. Arrows indicate the CD11b or IB4 or CD11b-IB4 positive cells. Scale bar=30μ. (B) Histograms show the quantitation of IB4-positive microglia in the ipsilateral cortex, hippocampus and thalamus 24 or 48 hours after TBI. In all three regions, the number of IB4-positive cells was significantly increased 24 h after TBI compared to sham animals. * p<0.001; * p<0.05; compared to sham. #p<0.05, ##p<0.001 compared to 24H TBI.

FIG. 4 shows that CCL20 is up-regulated in spleen 24 hours after mild TBI. PCR super array analysis was performed to analyze the gene expression in spleen tissues following TBI. The histograms show the mRNA expressional changes of different cytokines, chemokines and their receptors 24 hours after TBI. (A) shows up-regulated genes: CCL20 mRNA increased 5-fold in TBI animals compared to the sham animals. (B) The down-regulated genes with 2-fold or more down-regulation.

FIG. 5 shows that CCL20 expression is up-regulated in spleen and thymus after mild TBI. (A) Low magnification (scale bar=500μ) photomicrographs show the immunohistochemical labelling of CCL20 in spleen and thymus tissues in sham, and 24 h or 48 h after TBI. High magnification (scale bar=20μ) images of the selected areas from each section are shown in the inset of the corresponding image. (B) CCL20 immunoreactivity in spleen (left) and thymus (right) in sham or TBI animals was calculated using Image J program and expressed as mean area±S.E.M. CCL20 immunoreactivity increased significantly 24 h and 48 h after TBI compared to sham animals. *p<0.05. **p<0.001 compared to sham. (C) The histograms show the changes of CCL20 expression in spleen and thymus 24 or 48 hours post-TBI. ELISA was performed with rat anti-CCL20 antibody using a Duo set ELISA kit from R&D systems. In both tissues, CCL20 expression increased significantly 24 h alter TBI. *p<0.05, ** p<0.001 compared to sham animals.

FIG. 6 shows that CCL20 is expressed in rat brain cortex and hippocampus 48 h after TBI. (A) Immunostaining with anti-CCL20 antibody shows CCL20-expressing cells in cortex and hippocampus 48 h after TBI. Low magnification (scale bar 500=μ) photomicrographs with high magnification (scale bar 500=μ) images from selected areas shown in the inset. The immunostaining was localized in the pyknotic cell bodies (arrows) devoid of surrounding tissues indicating tissue damage. This immunostaining was not evident 24 h after TBI. Arrows indicate the CCL20-expressing cells. (B) CCL20-positive neurons in ipsilateral cortex and hippocampus were counted using the NIH Image J program and compared with corresponding areas from sham animals. CCL20 expression significantly increased in TBI animals 48 hours after impact. **p<0.001 compared to sham.

FIG. 7 shows that CCL20 expression is observed in the areas of neurodegeneration of cortex and hippocampus 48 hours after TBI. (A) High magnification photomicrographs of brain sections from animals subjected to TBI and sacrificed 24 or 48 h post-impact were stained with Fluoro-Jade or anti-CCL20 antibody. Fluoro-Jade staining was observed in the cortex and in the hippocampal CA1 and CA3 pyramidal cell layers 24 and 48 hours after TBI. While no CCL20 immunoreactivity was observed in the same regions of adjacent sections 24 h after TBI, CCL20 immunoreactivity was observed in the conical neurons as well as within the hippocampal CA1 and CA3 pyramidal cell layers at 48 h. FJ, Fluoro Jade. Scale bar=50μ. (B) Representative photomicrographs showing the FJ-CCL20 double staining in the cortex. CCL20 immunoreactivity was observed in most of the degenerating neurons (FJ positive) as indicated by arrows. CCL20 immunoreactivity was also observed in other cells that were not FJ positive. Scale bar=100μ.

FIG. 8 shows that CCL20 is expressed in rat brain cortical neurons 48 h after TBI. Fluorescence microscopic images double immunostained with anti-CCL20 antibody and the neuronal marker NeuN antibody showed that most of the CCL20-expressing cells in the cortex were also NeuN positive. White arrows indicate CCL20 positive neurons. Blue arrows indicate CCL20 positive non-neuronal cells. Scale bar=100μ.

FIG. 9 shows that immediate splenectomy reduces TBI-induced neurodegeneration and CCL20 expression in the cortex. Degenerating neurons (FJ positive) were observed 24 hours or 48 hours after the induction of LFPI in animals with immediate splenectomy (splenectomy group) or without immediate splenectomy (no splenectomy group). (A) The histograms show the estimation of FJ-positive neurons as quantitated by the Image 1 program in the cortex. (B) CCL20 expression was observed in the cortex 48 hours after LFPI in animals with immediate splenectomy (splenectomy group) or without immediate splenectomy (no splenectomy group). The histograms show the estimation of CCL20-positive cells in the cortex. ** p<0.0001 and * p<0.001 compared to sham animals within the group. #p<0.001 compared to 24 h or 48 h TBI between the groups.

FIG. 10 shows hippocampal neurodegeneration and CCL20 expression following LFPI. Animals subjected to LFPI were euthanized either 24 or 48 h post-insult. Brain sections from the animals were stained with Fluoro-Jade (A,C,E,G) or anti-CCL20 (B,D,F,H) to determine whether CCL20 is associated with cellular injury. Fluoro-Jade staining was prominent 24 h after insult and localized to the CA1 (A) and CA3 (E) pyramidal cell layers of the hippocampus, while CCL20 immunoreactivity was absent from the same regions in adjacent sections (B,F). Neurons labeled intensely with CCL20 within the CA1 (D) and CA3 (H) pyramidal cell layers at 48 h, when neurodegeneration was no longer evident (C,G). Quantification of CCL20 (1) demonstrated significant increases in total hippocampal chemokine expression at 48 h relative to sham-operated and 24 h animals, reflecting upregulated expression in both CA1 and CA3 (p<0.05). Scale bars=100 μm. Asterisk denotes significance from sham-operated controls. Pound sign denotes significance from 24 h LFPI.

FIG. 11 shows CCL20 upregulation in white matter following LFPI. Briefly, animals subjected to LFPI were euthanized either 24 or 48 h post-insult. Immunohistochemistry was performed on brain sections of the animals. (A) CCL20 immunoreactivity was present in the external capsule of rats euthanized 24 h after insult. (B) Sections from animals euthanized at 48 h showed intense, ubiquitous cellular CCL20 expression. CCL20 immunoreactivity was distributed in intrafasicular rows that are characteristic of oligodendrocytes (OLs). (C) Quantification of CCL20 immunoreactivity showed a significant increase at 48 h relative to sham-operated and 24 h animals (p<0.05). Scale bars=100 μm. Dashed line represents the division between the external capsule and adjacent cortical region. Asterisk denotes significance from sham-operated and 24 h rats.

FIG. 12 shows that CCL20 elicits cellular toxicity to neurons and OLs in vitro. Briefly, primary neuronal or OL cultures were subjected to oxygen glucose deprivation (OGD) in the presence or absence of 200 ng recombinant CCL20. Application of CCL20 increased LDH release in OL cultures under normoxic conditions and further enhanced OGD-induced cellular toxicity (p<0.05). The addition of CCL20 increased lactate dehydrogenase (LDH) release in neurons subjected to OGD relative to OGD alone (p<0.05), but had no effect on normoxic neuronal cultures. Asterisk denotes significance from normoxia. Pound sign denotes significance from OGD. NM=normoxia. OGD=oxygen glucose deprivation. a.u.=arbitrary units.

### DETAILED DISCLOSURE

The subject invention pertains to the use of CC chemokine ligand 20 (CCL20, also known as macrophage inflammatory protein 3α (MIP3α)) as a novel biomarker for early detection of traumatic brain injury and/or neurodegeneration in the brain. Advantageously, the diagnostic method of the subject invention allows for early detection of TBI within 48 hours, or even within 24 hours of the suspected primary injury. Additionally, the subject diagnostic method is sensitive, reliable, and easy-to-perform. The subject invention also provides treatment methods for traumatic brain injury by modulating systemic and/or brain-specific CCL20-CCR6 signaling. Also provided are uses of CCL20-CCR6 signaling as a target for screening for therapeutic agents that are useful for treatment of traumatic brain injury and/or neurodegeneration in the brain.

CCL20 interacts specifically with CC chemokine receptor 6 (CCR6) and one of the early chemokine-receptor combinations. CCL20 signaling attracts dendritic cells, T-cells and B-cells, and plays a significant role in inflammatory reactions [10]. Ohta et al. [11] have shown that CCL20 was upregulated under normothermic condition in a rat middle cerebral artery occlusion (MCAO) model. CCL20 is also expressed in inflamed epithelial cells [12] and in the synovial tissues of rheumatoid arthritis patients [13, 14]. Specifically, CCL20 and its receptor CCR6 are constitutively expressed in the choroid plexus of mice and human [18]. In the central nervous system, CCL20, a dual-acting chemokine that can inhibit immune reaction and attract inflammatory effectors and activators [16], is produced by astrocytes in response to bacterial infections [17]. Upregulation of CCL20 along with other cytokines has also been observed in human subjects one day after severe traumatic brain injury [15]. However, prior to the subject invention, it was unknown whether CCL20 is involved in systemic or brain-specific inflammatory response following TBI.

In the subject invention, it is discovered that approximately 80% of the neural injury in rats subjected to mild LFPI occurs at 24 and 48 hours post-insult, which represent two time points within the delayed injury phase. A quantitative method of injury characterization was developed that identified reproducible, region-specific neurodegeneration in response to a mild pressure pulse.

Prior to the subject invention, little was known about the signaling mechanisms that promote cellular injury after the acute phase of TBI or the systemic pro-inflammatory mediators that promote secondary injury. To uncover this, the subject invention adopted the LFPI model of TBI and developed a methodology that results in mild, reproducible injury that can be quantified for screening for therapeutics.

Most individuals afflicted by TBI have mild injury. In the subject invention, the experiments are conducted using an LFPI model of TBI. The pressure pulses used in the subject invention (within the range of 2.0-2.2 atm) are generally considered to reflect moderate injury in the rat model; thus, the subject invention lends external validity with regards to clinical applications.

It is also discovered that the areas predominantly affected by TBI include the cerebral cortex, hippocampus, and thalamus. In addition, hippocampal damage was localized to the ipsilateral hemisphere, in contrast to cortical and thalamic degeneration that was detected in both brain hemispheres. Thus, selection of the hippocampus for injury assessments limits variation that arises from diffusely distributed cellular injury and allows for a more focused, precise quantification strategy.

Current treatment for TBI aims to block the secondary injury phase and/or facilitate plasticity and repair after the initial impact. One important characteristic of the secondary injury is the deprivation of oxygen and glucose in the brain.

The spleen is a reservoir of peripheral macrophages and other immune cells in the body, and splenic signaling contributes to injury of various tissues after ischemic insult. The spleen responds to injury in the brain by releasing stored immune cells in the bloodstream, which then infiltrate the brain and promote a secondary inflammatory response that enhances neurodegeneration [34]. It has been reported that removal of the spleen prior to ischemia reperfusion injury to liver is hepatoprotective [27]. Neutrophils produce reactive oxygen species. TNF-α and nitric oxide [28, 29] in response to ischemia reperfusion, causing damage to the liver as well as kidney, heart, lungs and intestine [30]. Removal of the spleen reduces [31] and protects against damage and subsequent inflammation that causes damage to other organs [32].

It is discovered that CCL20 expression is upregulated in the spleen 24 hours post LFPI indicating the initiation or persistence of a splenic signal that drives neural inflammation and cell death. CCL20 expression is also up-regulated in the thymus 24 hours after mild TBI. CCL20 induces chemotaxis of CD4+T cells through the activation of CCR6. The binding of CCL20 to CCR6 on Th1 or Th17 cells is critical for T cell infiltration into the CNS through the choroid plexus. Indeed. T cells have been detected within the CNS in other neural injury models characterized by a compromised blood brain barrier (BBB) and oxygen glucose deprivation (OGD). A leaky BBB, OGD, and infiltration of peripheral leukocytes into the brain are characteristics of TBI injury, and peripheral CCL20 signaling can be an important initiator of T cell chemotaxis and extravasation into the brain parenchyma.

The subject invention also discovered that CCL20 was not expressed in degenerating hippocampal pyramidal cell layers or cerebral white matter until 48 hours after the primary insult. Therefore, it is postulated that peripheral CCL20 signaling promotes neurodegeneration, and this temporal expression profile is consistent with the delayed CCL20 expression observed in the brain. The subject invention also showed that neurodegeneration was more prevalent at 24 hours post-TBI than at the 48-hour time point.

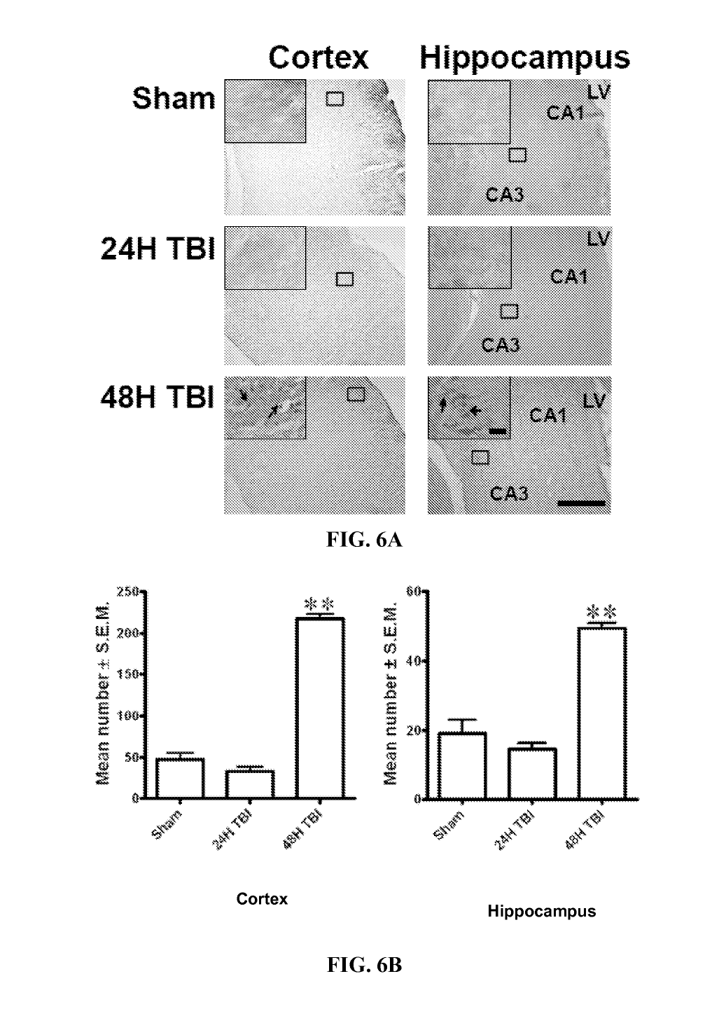

It is discovered that TBI induces increased neuronal expression of CCL20. Specifically, hippocampal neurons expressed CCL20 at 48 hours, which is 24 hours after the same cell layers showed prominent neurodegeneration. It is postulated that neuronal CCL20 expression can be a tombstone marker in cells that are beyond repair and need to be removed from the surrounding viable tissue. This explanation is consistent with the pyknotic morphology that was observed in CCL20-expressing neurons, as well as the fact that the areas surrounding the cell bodies appeared to be devoid of tissue (FIG. 6). During traumatic brain injury, infiltrating peripheral leukocytes promote cell death through the release of inflammatory cytokines. It is also postulated that CCL20—the 11 kDa protein could easily enter from the systemic circulation into the CNS through the compromised BBB and exert its effects in the absence of peripheral leukocytes. As a result, CCL20 levels in blood (e.g., whole blood, blood serum, and blood plasma), lymph, or cerebrospinal fluid can be utilized as an important diagnostic biomarker for determining the absence/presence of TBI injury and/or the severity of injury.

The morphological analysis, anatomical localization and co-localization with FJ and NeuN protein of CCL20-positive cells indicate that neurons represent the predominant cell type expressing CCL20 following TBI. It is also postulated that peroxysome proliferator-activated receptor g (PPARg) is down-regulated in neuronal cells after TBI.

CCL20 induces chemotaxis of CD4+T cells through activation of CCR6. This signaling interaction is critical for CNS infiltration of Th17+ cells through the choroid plexus. T cells have been detected within the CNS in other neural injury models characterized by a compromised BBB and OGD-related pathology. The results show that peripheral CCL20 signaling is an important initiator of T cell chemotaxis and extravasation into the brain parenchyma, and that CCL20 plays a role in apoptosis and pathological T cell responses that exacerbate tissue injury.

CCL20 plays an important role in neuroinflammation in brain tissue after TBI. In addition, peripheral CCL20 signaling causes, at least in part, the secondary phase of neural injury. The Examples also show that peripheral CCL20 signal mediates the neuropathological response to TBI, as CCL20 expression becomes elevated in the spleen and thymus prior to CCL20 elevation in brain tissue.

The subject invention incorporates by reference the entire disclosures of Das et al., Lateral fluid percussion injury of the brain inducers CCL20 inflammatory chemokine expression in rats. J. Neuroinflammation 2011, 8:148 [33].

Diagnosis of Traumatic Brain Injury and/or Neurodegeneration in the Brain

One aspect of the subject invention provides methods for diagnosing traumatic brain injury (TBI) and/or neurodegeneration in the brain. Advantageously, the subject invention allows for early detection of TBI within 48 hours, or even within 24 hours of the suspected primary injury.

In one embodiment, the subject invention provides a method for diagnosing traumatic brain injury and/or neurodegeneration in the brain, comprising:

a) obtaining a biological sample from the subject;

b) determining CC chemokine ligand 20 (CCL20) level in the biological sample; and

c) characterizing the subject's CCL20 level.

In one embodiment, traumatic brain injury can be detected by comparing the subject's CCL20 level to a predetermined reference value. In one embodiment, an elevated CCL20 level in the subject's biological sample, when compared to the predetermined reference value, indicates that the subject has traumatic brain injury and/or neurodegeneration in the brain. In one embodiment, the predetermined reference value for CCL20 is the CCL20 level present in corresponding biological samples obtained from a normal population that do not have brain injury. In one embodiment, the normal population do not have neural injury. In one embodiment, the normal population do not have inflammation or abnormal immune/autoimmune conditions. It is postulated that the level of CCL20 in a biological sample correlates to the severity of the secondary injury (e.g., neuroinflammation and/or neurodegeneration in brain tissue) caused by traumatic brain injury. In one embodiment, the degree of elevation in CCL20 level in the subject's biological sample, when compared to the predetermined reference value, indicates the severity of the secondary injury (e.g., neuroinflammation and/or neurodegeneration in brain tissue) caused by TBI.

The term “subject,” as used herein, describes an organism, including mammals such as primates. Mammalian species that can benefit from the subject methods include, but are not limited to, apes, chimpanzees, orangutans, humans, monkeys; and other animals such as dogs, cats, horses, cattle, pigs, sheep, goats, chickens, mice, rats, guinea pigs, and hamsters. Typically, the subject is a human.

In one embodiment, the subject is suspected of having traumatic brain injury and/or neurodegeneration in the brain. In one embodiment, a subject suspected of having traumatic brain injury has received primary injury, such as head injury. Primary injury can be caused by, for example, application of mechanical force to the head, car accidents, falls, sudden acceleration, concussion, and closed or penetrating head injury caused by weapons, firearms, and/or explosion.

The term “biological sample,” as used herein, includes but is not limited to a sample containing tissues, cells, and/or biological fluids isolated from a subject. Examples of biological samples include, but are not limited to, tissues, cells, biopsies, blood, lymph, serum, plasma, urine, saliva, and tears. In one embodiment, the biological sample is a blood (e.g., whole blood, blood serum, and blood plasma), lymph, or cerebrospinal fluid sample. In one embodiment, the biological sample is a spleen tissue sample. In one embodiment, the biological sample is a thymus tissue sample. In one embodiment, the biological sample is a brain tissue sample. In one embodiment, the biological sample is isolated from brain tissue that received primary injury (e.g., brain tissue damaged by open or penetrating head injury). In certain embodiments, the biological sample is a brain tissue sample isolated from the cerebral cortex, hippocampus (including CA1, CA2, CA3, and CA4) and/or thalamus tissue. In one embodiment, the biological sample is a brain tissue sample isolated from hippocampus CA1 and/or CA3. In a specific embodiment, the biological sample is a blood (e.g., whole blood, blood serum, and blood plasma) sample.

In one embodiment, the biological sample, such as the blood (e.g., whole blood, blood serum, and blood plasma), lymph, cerebrospinal fluid, spleen tissue, thymus tissue, and/or brain tissue sample, is obtained from a subject within 6, 12, 18, 24, 30, 36, or 48 hours after the subject received the primary injury. In another embodiment, the biological sample, such as the blood (e.g., whole blood, blood serum, and blood plasma), lymph, cerebrospinal fluid, spleen tissue, thymus tissue, and/or brain tissue sample, is obtained from a subject 24, 30, 36, 48, or 60 hours after the subject received the primary injury.

CCL20 level, as used herein, includes nucleic acid and protein levels of CCL20. In one embodiment, CCL20 level is CCL20 mRNA level. In another embodiment, CCL20 level is CCL20 protein level. In one embodiment, the subject invention involves the determination of human CCL20 nucleic acids and/or protein level. In one embodiment, the subject invention involves the determination of nucleic acid and/or protein level of human CCL20 isoform 1 (GenBank Accession No. NP_{—}004582.1; SEQ ID NO:1) and/or human CCL20 isoform 2 (GenBank Accession No. NP_{—}001123518.1; SEQ ID NO-2). Methods for detecting biomarkers (e.g., protein and nucleic acids) of the subject invention are well known in the art, including but not limited to, Western blots, Northern blots. Southern blots, enzyme-linked immunosorbent assay (ELISA), microarray, immunoprecipitation, immunofluorescence, immunocytochemistry, radioimmunoassay, polymerase chain reaction (PCR), real-time PCR, nucleic acid hybridization techniques, nucleic acid reverse transcription methods, nucleic acid amplification methods, and any combination thereof.

In one embodiment, the CCL20 level in the biological sample is determined by contacting a sample with an agent selected from:

(a) antibodies that specifically bind to CCL20 or antibody fragments thereof, CCL20 binding partners, or aptamers that specifically bind to CCL20; or

(b) oligonucleotides that are partially or fully complementary to, and bind to, nucleic acid sequences encoding CCL20 proteins.

In one embodiment, the subject invention can detect traumatic brain injury before the subject exhibits detectable level of neuroinflammation and/or neurodegeneration in brain tissue. In one embodiment, the subject invention can detect traumatic brain injury before the subject exhibits TBI symptoms, such as for example, impairment in speech, motor ability, coordination, cognitive ability, memory, and/or learning.

In one embodiment, the subject invention can detect mild and/or moderate TBI within 12, 18, 24, or 48 hours of the suspected primary injury. TBI can be classified based on severity of the injury. In one embodiment, the severity of TBI is classified based on the Glasgow Coma Scale (GCS), wherein mild TBI has a GCS of 13 or above, moderate GCS has a GCS of 9-12, and severe TBI has a GCS of 8 or lower.

In one embodiment, the subject invention provides a method for diagnosing whether a subject has traumatic brain injury, comprising:

a) obtaining a biological sample from the subject;

b) determining CC chemokine ligand 20 (CCL20) level in the biological sample; and

c) comparing the CCL20 level in the biological sample of the subject to a predetermined reference value, and diagnosing the subject as having traumatic brain injury if the CCL20 level in the biological sample of the subject is higher than the predetermined reference value.

In a further embodiment, the biological sample is a blood sample, and the method further comprises treating the subject if the subject is diagnosed of having traumatic brain injury.

In one embodiment, the subject invention provides a method for diagnosing whether a subject has neurodegeneration in the brain following a suspected primary injury, wherein the method comprises:

a) obtaining a biological sample from the subject;

b) determining CC chemokine ligand 20 (CCL20) level in the biological sample; and

c) comparing the CCL20 level in the biological sample of the subject to a predetermined reference value, and diagnosing the subject as having neurodegeneration in the brain if the CCL20 level in the biological sample of the subject is higher than the predetermined reference value.

In one embodiment, the subject method for diagnosing traumatic brain injury and/or neurodegeneration in the brain further comprises: determining level of one or more second biomarkers in a subject, and characterizing said level of the second biomarker(s). The biomarkers useful according to the subject invention include, but are not limited to, CCL24, CCL6, CCR1, CCR2, CCR3, CX3CL1, CXCL12, CXCL6, IL1F5, IL1R2, ITGB2, PF4, TNFRSF1b, Cd401g, Tollip, XCR1, CCL12, CCL19, CCL22, CCL7, CCR8, CRP, CXCL12, CXCL9, LFNG, IL3, IL4, and IL8RA. In one embodiment, the level of the biomarkers is determined before, during, or after the determination of CCL20 level in a subject. Optionally, the determination is made at multiple times to monitor the change over time.

In one embodiment, an elevated level of one or more biomarkers selected from CCL24, CCL6, CCR1, CCR2, CCR3, CX3CL1, CXCL12, CXCL6, IL1F5, IL1R2, ITGB2, PF4, TNFRSF1b, Cd401g, Tollip, and XCR1 in the subject's biological sample, when compared to the predetermined reference value, indicates that the subject has traumatic brain injury and/or neurodegeneration in the brain. In one embodiment, a decreased level of one or more biomarkers selected from CCL12, CCL19, CCL22, CCL7, CCR8, CRP, CXCL12, CXCL9, LFNG, IL3, IL4, and IL8RA in the subject's biological sample, when compared to the predetermined reference value, indicates that the subject has traumatic brain injury and/or neurodegeneration in the brain. In one embodiment, the predetermined reference value for the second biomarker is the level of said biomarker present in corresponding biological samples obtained from a normal population that do not have brain injury or neurodegeneration.

In one embodiment, various brain imaging techniques (e.g., CAT scan, MRI, SPECT and/or PET scan) can be used to aid the detection of the location of primary brain injury. In addition, neuropyschological and physical testing can be conducted to aid the determination of presence and/or the severity of traumatic brain injury.

### Biological Assays and Assay Kits

Another aspect of the invention provides probes and kits suitable for diagnosing traumatic brain injury and/or neurodegeneration in the brain. In one embodiment, the subject invention provides a diagnostic probe or kit comprising an agent that binds specifically to a CCL20 protein or comprising a nucleic acid molecule encoding a CCL20 protein.

In one specific embodiment, the diagnostic probe or kit comprises:

(a) an antibody that specifically binds to CCL20 or an antibody fragment thereof, a CCL20 binding partner, or an aptamer that specifically binds to CCL20; and/or

(b) an oligonucleotide complementary to a nucleic acid sequence encoding a CCL20 protein, an oligonucleotide complementary to a fragment of a nucleic acid sequence encoding a CCL20 protein, or an oligonucleotide that binds specifically to a nucleic acid sequence encoding a CCL20 protein.

The invention also concerns an array that may be used to assess level of biomarkers of interest within a sample in accordance with the treatment and diagnostic methods of the invention.

The substrate may be any suitable support for the capture probes that may be contacted with a sample. The substrate may be any solid or semi-solid carrier for supporting the capture probes, such as a particle (e.g., magnetic or latex particle), a microtiter multi-well plate, a bead, a slide, a filter, a chip, a membrane, a cuvette, or a reaction vessel.

In certain embodiments, the samples are assayed for assessing one or more biomarkers of the invention. The biomarker and biomarkers useful according to the subject invention (e.g., CCL20, CCL24, CCL6, CCR1, CCR2, CCR3, CX3CL1, CXCL12, CXCL6, IL1F5, IL1R2, ITGB2, PF4, TNFRSF1b, Cd401g, Tollip, XCR1, CCL12, CCL19, CCL22, CCL7, CCR8, CRP, CXCL12, CXCL9, LFNG, IL3, IL4, and IL8RA) can be determined by methods including, but not limited to, enzyme-linked immunosorbant assays (ELISA), Western blot, immunological assays, microarrays, radioimmunoassays (RIAs), lateral flow assays, immunochromatographic strip assays, automated flow assays, immunoprecipitation assays, reversible flow chromatographic binding assays, agglutination assays, Southern blots, immunofluorescence, flow cytometry, immunocytochemistry, nucleic acid hybridization techniques, nucleic acid reverse transcription methods, nucleic acid amplification, polymerase chain reaction (PCR), DNA arrays, protein arrays, mass spectrometry, and any combination thereof.

The level and/or the presence of the biomarkers can be determined either at the nucleic acid (such as mRNA) or protein level. In some embodiments, the expression of a biomarker is detected on a protein level using, for example, antibodies that are directed against specific biomarker proteins. These antibodies can be used in various methods such as Western blot, ELISA, immunoprecipitation, immunocytochemistry, flow cytometry, and cell sorting (FACS). Reduction in biomarker gene expression can be detected at the mRNA level by techniques including, but not limited to, real-time RT-PCR, microarray analysis, and Northern blotting. Preferably, all expression data is compared with levels of a “house keeping” gene to normalize for variable amounts of RNA in different samples.

In one embodiment of the method of the invention, the determining step comprises: (a) contacting the sample with a binding agent that binds biomarker protein to form a complex; (b) detecting the complex; and (c) correlating the detected complex to the amount of biomarker protein in the sample. In a specific embodiment, the detecting of (b) further comprises linking or incorporating a label onto the agent, or using ELISA-based immunoenzymatic detection.

In another embodiment of the method of the invention, the determining step comprises: (a) contacting the sample with a binding agent that binds biomarker nucleic acid (e.g., mRNA) to form a complex; (b) detecting the complex; and (c) correlating the detected complex to the amount of biomarker nucleic acid in the sample. In a specific embodiment, the detecting of (b) further comprises linking or incorporating a label onto the agent, or using ELISA-based immunoenzymatic detection.

The terms “detecting” or “detect” include assaying or otherwise establishing the presence or absence of the target biomarker, subunits thereof, or combinations of agent bound targets, and the like. The term encompasses quantitative, semi-quantitative, and qualitative detection methodologies. Embodiments of the invention involve detection of biomarker protein (as opposed to nucleic acid molecules encoding biomarker protein). In one embodiment the detection method is an ELISA-based method. Preferably, in the various embodiments of the invention, the detection method provides an output (i.e., readout or signal) with information concerning the presence, absence, or amount of the biomarker in a sample. For example, the output may be qualitative (e.g., “positive” or “negative”), or quantitative (e.g., a concentration such as nanograms per milliliter).

In one embodiment, the assessing step comprises the following steps:

(a) incubating a biological sample with a first antibody specific for the biomarker protein (CCL24, CCL6, CCR1, CCR2, CCR3, CX3CL1, CXCL12, CXCL6, IL1F5, IL1R2, ITGB2, PF4, TNFRSF1b, Cd401g, Tollip, XCR1, CCL12, CCL19, CCL22, CCL7, CCR8, CRP, CXCL12, CXCL9, LFNG, IL3, IL4, and IL8RA) which is directly or indirectly labeled with a detectable substance, and a second antibody specific for the first antibody;

(b) separating the first antibody from the second antibody to provide a first antibody phase and a second antibody phase;

(c) detecting the detectable substance in the first or second antibody phase thereby quantitating the biomarker in the sample; and

(d) comparing the quantitated biomarker level with a standard.

As is known in the art, polypeptides or proteins in test samples are commonly detected with immunoassay devices and methods. Alternatively, or additionally, aptamers can be selected and used for binding of even greater specificity, as is well known in the art. These devices and methods can utilize labeled molecules in various sandwich, competitive, or non-competitive assay formats, to generate a signal that is related to the presence or amount of an analyte of interest. Additionally, certain methods and devices, such as biosensors and optical immunoassays, may be employed to determine the presence or amount of analytes without the need for a labeled molecule.

Specific immunological binding of the antibody to the biomarker can be detected directly or indirectly. Direct labels include fluorescent or luminescent tags, metals, dyes, radionuclides, and the like, attached to the antibody. Indirect labels include various enzymes well known in the art, such as alkaline phosphatase, horseradish peroxidase and the like.

The antibody-based assays can be considered to be of four types: direct binding assays, sandwich assays, competition assays, and displacement assays. In a direct binding assay, either the antibody or antigen is labeled, and there is a means of measuring the number of complexes formed. In a sandwich assay, the formation of a complex of at least three components (e.g., antibody-antigen-antibody) is measured. In a competition assay, labeled antigen and unlabelled antigen compete for binding to the antibody, and either the bound or the free component is measured. In a displacement assay, the labeled antigen is pre-bound to the antibody, and a change in signal is measured as the unlabelled antigen displaces the bound, labeled antigen from the receptor.

The use of immobilized antibodies specific for the biomarkers is also contemplated by the subject invention and is well known by one of ordinary skill in the art. The antibodies can be immobilized onto a variety of solid supports, such as magnetic or chromatographic matrix particles, the surface of an assay place (such as microtiter wells), pieces of a solid substrate material (such as plastic, nylon, paper), and the like. An assay strip can be prepared by coating the antibody or a plurality of antibodies in an array on solid support. This strip can then be dipped into the test sample and then processed quickly through washes and detection steps to generate a measurable signal, such as a colored spot.

The analysis of a plurality of biomarkers may be carried out separately or simultaneously with one test sample. Several biomarkers may be combined into one test for efficient processing of a multiple of samples. In addition, one skilled in the art would recognize the value of testing multiple samples (for example, at successive time points) from the same individual. Such testing of serial samples will allow the identification of changes in biomarker levels over time.

The analysis of biomarkers can be carried out in a variety of physical formats as well. For example, the use of microtiter plates or automation can be used to facilitate the processing of large numbers of test samples. Particularly useful physical formats comprise surfaces having a plurality of discrete, addressable locations for the detection of a plurality of different analytes. Such formats include protein microarrays, or “protein chips” (see, e.g., Ng and Ilag, J. Cell Mol. Med. 6: 329-340 (2002)) and capillary devices.

In one embodiment, the determining step in the assays (methods) of the invention can involve contacting, combining, or mixing the sample and the solid support, such as a reaction vessel, microvessel, tube, microtube, well, multi-well plate, or other solid support.

The methods of the invention can be carried out on a solid support. The solid supports used may be those which are conventional for the purpose of assaying an analyte in a sample, and are typically constructed of materials such as cellulose, polysaccharide such as Sephadex, and the like, and may be partially surrounded by a housing for protection and/or handling of the solid support. The solid support can be rigid, semi-rigid, flexible, elastic (having shape-memory), etc., depending upon the desired application. The biomarkers can be accessed in a sample in vivo or in vitro (er vivo).

Samples and/or binding agents may be arrayed on the solid support, or multiple supports can be utilized, for multiplex detection or analysis.

In another embodiment, the subject invention provides a kit for the analysis of biomarkers. Such a kit preferably comprises devices and reagents for the analysis of at least one test sample and instructions for performing the assay. The kit may contain aptamers specific for a target biomarker. Optionally the kits may contain one or more means for using information obtained from immunoassays performed for a biomarker panel. Biomarker antibodies or antigens may be incorporated into immunoassay kits depending upon which biomarker autoantibodies or antigens are being measured. A first container may include a composition comprising an antigen or antibody preparation. Both antibody and antigen preparations should preferably be provided in a suitable titrated form, with antigen concentrations and/or antibody titers given for easy reference in quantitative applications.

The kits may also include an immunodetection reagent or label for the detection of specific immunoreaction between the provided antigen and/or antibody, as the case may be, and the sample. Suitable detection reagents are well known in the art as exemplified by radioactive, enzymatic or otherwise chromogenic ligands, which are typically employed in association with the antigen and/or antibody, or in association with a second antibody having specificity for first antibody. Thus, the reaction is detected or quantified by means of detecting or quantifying the label. Immunodetection reagents and processes suitable for application in connection with the novel methods of the subject invention are generally well known in the art.

The reagents may also include ancillary agents such as buffering agents and protein stabilizing agents, e.g., polysaccharides and the like. The kit may further include where necessary agents for reducing background interference in a test, agents for increasing signal, software and algorithms for combining and interpolating biomarker values to produce a prediction of clinical outcome of interest, apparatus for conducting a test, calibration curves and charts, standardization curves and charts, and the like.

As used herein, the term “antibody” refers to an intact immunoglobulin having two light and two heavy chains or any antibody fragments thereof sufficient to bind a target of interest. Thus a single isolated antibody or antibody fragment may be a polyclonal antibody, a high affinity polyclonal antibody, a monoclonal antibody, a synthetic antibody, a recombinant antibody, a chimeric antibody, a humanized antibody, or a human antibody.

The term “antibody fragment,” as used herein, refers to less than an intact antibody structure, including, without limitation, an isolated single antibody chain, an Fv construct, a Fab construct, a light chain variable or complementarity determining region (CDR) sequence, etc. A recombinant molecule bearing the binding portion of an antibody, e.g., carrying one or more variable chain CDR sequences that hind the biomarker, may also be used in the detection assay of this invention.

Other reagents for the detection of protein in samples, such as peptide mimetics, synthetic chemical compounds capable of detecting the biomarker may be used in other assay formats for the quantitative detection in samples, such as Western blots, flow cytometry, etc.

As indicated above, kits of the invention include reagents for use in the methods described herein, in one or more containers. The kits may include primers, specific internal controls, and/or probes, buffers, and/or excipients, separately or in combination. Each reagent can be supplied in a solid form or liquid buffer that is suitable for inventory storage. Kits may also include means for obtaining a sample from a host organism or an environmental sample.

Kits of the invention can be provided in suitable packaging. As used herein, “packaging” refers to a solid matrix or material customarily used in a system and capable of holding within fixed limits one or more of the reagent components for use in a method of the subject invention. Such materials include glass and plastic (e.g., polyethylene, polypropylene, and polycarbonate) bottles, vials, paper, plastic, and plastic-foil laminated envelopes and the like. Preferably, the solid matrix is a structure having a surface that can be derivatized to anchor an oligonucleotide probe, primer, molecular beacon, specific internal control, etc. Preferably, the solid matrix is a planar material such as the side of a microtiter well or the side of a dipstick. In certain embodiments, the kit includes a microtiter tray with two or more wells and with reagents including primers, probes, specific internal controls, and/or molecular beacons in the wells.

“Specific binding” or “specificity” refers to the ability of an antibody or other agent to detectably bind an epitope presented on an antigen, while having relatively little detectable reactivity with other proteins or structures. Specificity can be relatively determined by binding or competitive binding assays, using, e.g., Biacore instruments. Specificity can be exhibited by, e.g., an about 10:1, about 20:1, about 50:1, about 100:1, 10.000:1 or greater ratio of affinity/avidity in binding to the specific antigen versus nonspecific binding to other irrelevant molecules.

“Selectivity” refers to the preferential binding of a protein to a particular region, target, or peptide as opposed to one or more other biological molecules, structures, cells, tissues, etc. For example, selectivity can be determined by competitive ELISA or Biacore assays. The difference in affinity/avidity that marks selectivity can be any detectable preference (e.g., a ratio of more than 1:1.1, or more than about 1:5, if detectable.

Treatment of Traumatic Brain Injury and/or Neurodegeneration in the Brain

Another aspect of the invention provides methods for treatment of traumatic brain injury and/or neurodegeneration in the brain. In one embodiment, the method comprises modulating CCL20 level in a subject who has TBI. In one embodiment, the method modulates/reduces CCL20 level in spleen, blood, lymph, thymus, and/or brain tissue.

In one embodiment, the method comprises administering to a subject who has TBI an effective amount of a therapeutic agent that reduces CCL20 level. In certain embodiments, therapeutic agents for treatment of traumatic brain injury and/or neurodegeneration in the brain include, but are not limited to, an agent that binds specifically to CCL20 proteins or nucleic acids encoding CCL20 proteins. Inhibitors of CCL20 useful according to the subject invention include, but are not limited to, anti-CCL20 antibodies and CCL20 antagonists.

In certain embodiments, the levels of CCL20 expression, the pro-inflammatory activity of CCL20, or the binding of CCL20 to CCR6 in the blood, lymph, spleen and/or thymus tissue, and/or neuronal cells (e.g., neuronal cells in the brain) are reduced in accordance with the treatment method of the subject invention. In certain embodiments, the expression level of CCL20 in the cerebral cortex, hippocampus, and/or thalamus of a TBI subject is reduced.

In one embodiment, the method for treating traumatic injury and/or neurodegeneration in the brain comprises modulating and/or inhibiting pro-inflammatory CCL20 signaling in a subject. In one embodiment, the method reduces the level, activity, and/or expression of C-C chemokine receptor type 6 (CCR6). In one embodiment, the method modulates or inhibits binding of CCL20 to CCR6. In one embodiment, the method comprises administering to a subject an effective amount of a therapeutic agent that modulates or reduces the level, activity, and/or expression of CCR6. In one embodiment, the method comprises administering to a subject an effective amount of a therapeutic agent that inhibits binding of CCL20 to CCR6. Inhibitors of CCR6 useful according to the subject invention include, but are not limited to, anti-CCR6 antibodies and CCR6 antagonists.

In one embodiment, the subject is diagnosed with TBI and/or neurodegeneration in the brain. In one embodiment, the subject has elevated CCL20 level in a biological sample, such as for example, a sample obtained from spleen, blood (e.g., whole blood, blood serum, blood plasma), lymph, thymus, cerebrospinal fluid, and/or brain tissue.

In a further embodiment, the method for treating TBI comprises the administration of an effective amount of an anti-inflammatory and/or a neuroprotective agent.

The term “treatment” or any grammatical variation thereof (e.g., treat, treating, and treatment etc.), as used herein, includes but is not limited to, ameliorating or alleviating a symptom of a disease or condition, reducing, suppressing, inhibiting, lessening, or affecting the progression and/or severity of an undesired physiological change or a diseased condition. For instance, treatment includes reducing or ameliorating the secondary injury (e.g., neuroinflammation and/or neurodegeneration in brain tissue) caused by TBI.

The term “effective amount,” as used herein, refers to an amount that is capable of treating or ameliorating a disease or condition or otherwise capable of producing an intended therapeutic effect. In certain embodiments, the effective amount enables a 5%, 10%, 20%, 30%, 40%, 50%, 75%, 90%, 95%, 99% or 100% reduction in CCL20 level in a biological sample. In another embodiment, the effective amount reduces or ameliorates the secondary injury (e.g., neuroinflammation and/or neurodegeneration in brain tissue) caused by TBI.

In certain embodiments, agents for treatment of TBI and/or neurodegeneration in the brain include, but are not limited to, anti-CCL20 antibodies, aptamers, CCL20 binding partners, and small molecule inhibitors of CCL20.

In one embodiment, the therapeutic agent for treating TBI and/or neurodegeneration in the brain is an antibody that binds specifically to CCL20. In a further specific embodiment, the therapeutic agent for treating TBI and/or neurodegeneration in the brain is an antibody that binds specifically to human CCL20. In some embodiments, therapeutic agents for treating TBI and/or neurodegeneration in the brain include antibodies that bind specifically to CCL20 proteins of non-human animals including, but not limited to, apes, chimpanzees, orangutans, monkeys, dogs, cats, horses, pigs, sheep, goats, mice, rats, and guinea pigs. The skilled artisan could easily construct CCL20-specific antibodies to specifically target any CCL20 proteins publically known. In a specific embodiment, the therapeutic agent for treating TBI and/or neurodegeneration in the brain is an antibody or aptamer that binds specifically to a human CCL20 of SEQ ID NO:1 or SEQ ID NO:2.

In certain embodiments, agents for treatment of TBI and/or neurodegeneration in the brain include, but are not limited to, anti-CCR6 antibodies, aptamers, CCR6 binding partners, and small molecule inhibitors of CCR6.

In one embodiment, the therapeutic agent for treating TBI and/or neurodegeneration in the brain is an antibody that binds specifically to CCR6. In a further specific embodiment, the therapeutic agent for treating TBI and/or neurodegeneration in the brain is an antibody that binds specifically to human CCR6. In some embodiments, therapeutic agents for treating TBI and/or neurodegeneration in the brain include antibodies that bind specifically to CCR6 proteins of non-human animals including, but not limited to, apes, chimpanzees, orangutans, monkeys, dogs, cats, horses, pigs, sheep, goats, mice, rats, and guinea pigs. The skilled artisan could easily construct CCR6-specific antibodies to specifically target any CCR6 proteins publically known.

In some embodiments, the therapeutic agent treating TBI and/or neurodegeneration in the brain is a siRNA having a sequence sufficiently complementary to a target CCL20 mRNA sequence to direct target-specific RNA interference (RNAi). In some embodiments, the therapeutic agent for treating TBI and/or neurodegeneration in the brain is siRNA having a sequence sufficiently complementary to a target human CCL20 mRNA sequence (such as mRNA encoding SEQ ID NO:1 or SEQ ID NO:2) to direct target-specific RNA interference.

In one embodiment, the subject invention provides a method for treating traumatic brain injury and/or neurodegeneration in the brain, wherein the method comprises reducing CCL20 expression by introducing into a cell an antisense molecule against CCL20.

In some embodiments, the therapeutic agent treating TBI and/or neurodegeneration in the brain is a siRNA having a sequence sufficiently complementary to a target CCR6 mRNA sequence to direct target-specific RNA interference (RNAi). In one embodiment, the subject invention provides a method for treating traumatic brain injury and/or neurodegeneration in the brain, wherein the method comprises reducing CCR6 expression by introducing into a cell an antisense molecule against CCR6.

In certain embodiments, antisense molecules against CCL20 and/or CCR6 are introduced into cells of the spleen, the thymus, and/or the brain (including neuronal cells of the brain regions including the cerebral cortex, the hippocampus (including hippocampal CA1 and CA3 pyramidal cell layers) and the thalamus) of a subject that has TBI.

Examples of antisense polynucleotides include, but are not limited to, single-stranded DNAs and RNAs that bind to complementary target the mRNA of interest (such as CCL20 and CCR6 mRNA) and inhibit translation and/or induce RNaseH-mediated degradation of the target transcript; siRNA oligonucleotides that target or mediate mRNA degradation; ribozymes that cleave mRNA transcripts; and nucleic acid aptamers and decoys, which are non-naturally occurring oligonucleotides that bind to and block protein targets in a manner analogous to small molecule drugs.

In a further specific embodiment, the therapeutic agent for treating TBI and/or neurodegeneration in the brain is an antisense molecule against to human CCL20 and/or CCR6 mRNA. In some embodiments, therapeutic agents for treating TBI and/or neurodegeneration in the brain include antisense molecules against CCL20 and/or CCR6 mRNA of non-human animals including, but not limited to, apes, chimpanzees, orangutans, monkeys, dogs, cats, horses, pigs, sheep, goats, mice, rats, and guinea pigs. The skilled artisan could easily construct antisense molecules against any CCL20 and/or CCR6 mRNA sequences publically known. In a specific embodiment, the therapeutic agent for treating TBI and/or neurodegeneration in the brain is an antisense molecule against a human CCL20 mRNA encoding the CCL20 protein of SEQ ID NO: 1 or SEQ ID NO:2. As will be required by those skilled in the art, the antisense molecule does not have to be full length to be effective.

The term “nucleotide” refers to a nucleoside having one or more phosphate groups joined in ester linkages to the sugar moiety. Exemplary nucleotides include nucleoside monophosphates, diphosphates and triphosphates. The terms “polynucleotide” and “nucleic acid molecule” are used interchangeably herein and refer to a polymer of nucleotides joined together by a phosphodiester linkage between 5′ and 3′ carbon atoms.

The terms “nucleic acid” or “nucleic acid sequence” encompass an oligonucleotide, nucleotide, polynucleotide, or a fragment of any of these, DNA or RNA of genomic or synthetic origin, which may be single-stranded or double-stranded and may represent a sense or antisense strand, peptide nucleic acid (PNA), or any DNA-like or RNA-like material, natural or synthetic in origin. As will be understood by those of skill in the art, when the nucleic acid is RNA, the deoxynucleotides A, G, C, and T are replaced by ribonucleotides A, G, C, and U, respectively.

As used herein, the term “RNA” or “RNA molecule” or “ribonucleic acid molecule” refers generally to a polymer of ribonucleotides. The term “DNA” or “DNA molecule” or deoxyribonucleic acid molecule” refers generally to a polymer of deoxyribonucleotides. DNA and RNA molecules can be synthesized naturally (e.g., by DNA replication or transcription of DNA, respectively). RNA molecules can be post-transcriptionally modified. DNA and RNA molecules can also be chemically synthesized. DNA and RNA molecules can be single-stranded (i.e., ssRNA and ssDNA, respectively) or multi-stranded (e.g., double stranded, i.e., dsRNA and dsDNA, respectively). Based on the nature of the invention, however, the term “RNA” or “RNA molecule” or “ribonucleic acid molecule” can also refer to a polymer comprising primarily (i.e., greater than 80% or, preferably greater than 90%) ribonucleotides but optionally including at least one non-ribonucleotide molecule, for example, at least one deoxyribonucleotide and/or at least one nucleotide analog.

As used herein, the term “nucleotide analog”, also referred to herein as an “altered nucleotide” or “modified nucleotide,” refers to a non-standard nucleotide, including non-naturally occurring ribonucleotides or deoxyribonucleotides. Preferred nucleotide analogs are modified at any position so as to alter certain chemical properties of the nucleotide yet retain the ability of the nucleotide analog to perform its intended function.

As used herein, the term “RNA interference” (“RNAi”) refers to a selective intracellular degradation of RNA. RNAi occurs in cells naturally to remove foreign RNAs (e.g., viral RNAs). Natural RNAi proceeds via fragments cleaved from free dsRNA which direct the degradative mechanism to other similar RNA sequences. Alternatively, RNAi can be initiated by the hand of man, for example, to silence the expression of endogenous target genes.

As used herein, the term “small interfering RNA” (“siRNA”) (also referred to in the art as “short interfering RNAs”) refers to an RNA (or RNA analog) comprising between about 10-50 nucleotides (or nucleotide analogs) which is capable of directing or mediating RNA interference.

As used herein, a siRNA having a “sequence sufficiently complementary to a target mRNA sequence to direct target-specific RNA interference (RNAi)” means that the siRNA has a sequence sufficient to trigger the destruction of the target mRNA (e.g., CCL20 mRNA) by the RNAi machinery or process. “mRNA” or “messenger RNA” or “transcript” is single-stranded RNA that specifies the amino acid sequence of one or more polypeptides. This information is translated during protein synthesis when ribosomes bind to the mRNA.

The subject invention also contemplates vectors (e.g., viral vectors) and expression constructs comprising the nucleic acid molecules useful for inhibiting CCL20 expression and/or activity. In an embodiment, the vector comprises a siRNA that targets CCL20 mRNA. In another embodiment, the vector comprises a nucleic acid molecule encoding an anti-CCL20 antibody.

As used herein, the term “expression construct” refers to a combination of nucleic acid sequences that provides for transcription of an operably linked nucleic acid sequence. As used herein, the term “operably linked” refers to a juxtaposition of the components described, wherein the components are in a relationship that permits them to function in their intended manner. In general, operably linked components are in contiguous relation.

Expression constructs of the invention will also generally include regulatory elements that are functional in the intended host cell in which the expression construct is to be expressed. Thus, a person of ordinary skill in the art can select regulatory elements for use in, for example, bacterial host cells, yeast host cells, mammalian host cells, and human host cells. Regulatory elements include promoters, transcription termination sequences, translation termination sequences, enhancers, and polyadenylation elements.

An expression construct of the invention can comprise a promoter sequence operably linked to a polynucleotide sequence encoding a peptide of the invention. Promoters can be incorporated into a polynucleotide using standard techniques known in the art. Multiple copies of promoters or multiple promoters can be used in an expression construct of the invention. In a preferred embodiment, a promoter can be positioned about the same distance from the transcription start site as it is from the transcription start site in its natural genetic environment. Some variation in this distance is permitted without substantial decrease in promoter activity. A transcription start site is typically included in the expression construct. In one embodiment, the subject method for treating traumatic brain injury and/or neurodegeneration in the brain further comprises: modulating the level of one or more second biomarkers selected from CCL24, CCL6, CCR1, CCR2, CCR3, CX3CL1, CXCL12, CXCL6, IL1F5, IL1R2, ITGB2, PF4, TNFRSF1b, Cd401g, Tollip, XCR1, CCL12, CCL19, CCL22, CCL7, CCR8, CRP, CXCL12, CXCL9, LFNG, IL3, IL4, and IL8RA in a subject who has traumatic brain injury. In one embodiment, the method for treating TBI and/or neurodegeneration in the brain further comprises modulating or reducing the level of one or more second biomarkers selected from CCL24, CCL6, CCR1, CCR2, CCR3, CX3CL1, CXCL12, CXCL6, IL1F5, IL1R2, ITGB2, PF4, TNFRSF1b, Cd401g, Tollip, and XCR1. In one embodiment, the method comprises administering to a subject who has TBI and/or neurodegeneration in the brain an effective amount of a therapeutic agent that reduces the level of CCL24, CCL6. CCR1, CCR2, CCR3, CX3CL1, CXCL12, CXCL6, IL1F5, IL1R2, ITGB2, PF4, TNFRSF1b, Cd401g, Tollip, and/or XCR1. Inhibitors of the above mentioned biomarkers include, but are not limited to, antibodies and antagonists of CCL24, CCL6, CCR1, CCR3, CX3CL1, CXCL12, CXCL6, IL1F5, IL1R2, ITGB2, PF4, TNFRSF1b. Cd401g, Tollip, and/or XCR1.

In another embodiment, the method for treating TBI and/or neurodegeneration in the brain further comprises modulating or increasing one or more second biomarkers selected from CCL12, CCL19, CCL22, CCL7, CCR8, CRP, CXCL12, CXCL9, LFNG, IL3, IL4, and IL8RA.

In one embodiment, the method comprises administering to a subject who has TBI and/or neurodegeneration in the brain an effective amount of a therapeutic agent that increases the level of CCL12, CCL19, CCL22, CCL7, CCR8, CRP, CXCL12, CXCL9, LFNG, IL3, IL4, and/or IL8RA. In one embodiment, the method comprises administering to a subject who has TBI and/or neurodegeneration in the brain an effective amount of CCL12, CCL19, CCL22, CCL7, CCR8, CRP, CXCL12, CXCL9, LFNG, IL3, IL4, and/or IL8RA. Another aspect of the subject invention pertains to use of CCL20-CCR6 signaling as a target for screening for therapeutics for traumatic brain injury and/or neurodegeneration in the brain. The therapeutic agent can be a drug, chemical, compound, protein or peptide, or a nucleic acid molecule (e.g., DNA, RNA such as siRNA).

In one embodiment, the screening method comprises:

a) administering a candidate molecule to an animal subject that received traumatic brain injury and/or neurodegeneration in the brain,

wherein the candidate molecule is selected from an agent that modulates or reduces the level of CCL20, an agent that modulates or reduces the level of CCR6, an agent that modulates or inhibits the binding of CCL20 to CCR6, an agent that modulates or inhibits CCR6 signaling, and an agent that modulates or inhibits the expression of CCL20 and/or CCR6;

b) determining the level of neuroinflammation and/or neurodegeneration in brain tissue of the animal subject; and

c) selecting the candidate molecule if said molecule reduces the level of neuroinflammation and/or neurodegeneration in brain tissue of the animal subject, when compared to that of a control animal subject that received the same brain injury but is untreated with said candidate molecule.

In another embodiment, the method for screening for therapeutics for TBI comprises:

a) administering a candidate molecule to an animal subject that received traumatic brain injury,

wherein the candidate molecule is selected from:

(i) an agent that modulates CCL24, CCL6, CCR1, CCR3, CX3CL1, CXCL12, CXCL6, IL1F5, IL1R2, ITGB2, PF4, TNFRSF1b, Cd401g, Tollip, XCR1, CCL2, CCL2, CCL9, CCL22, CCL7, CCR8, CRP, CXCL12, CXCL9, LFNG, IL3, IL4, and/or IL8RA, and

(ii) CCL24, CCL6, CCR1, CCR3, CX3CL1, CXCL12, CXCL6, IL1F5, IL1R2, ITGB2, PF4, TNFRSF1b, Cd401g Tollip, XCR1, CCL12, CCL19, CCL22, CCL7, CCR8, CRP, CXCL12, CXCL9, LFNG, IL3, IL4, and IL8RA;

b) determining the level of neuroinflammation and/or neurodegeneration in brain tissue of the animal subject; and

c) selecting the candidate molecule if said molecule reduces the level of neuroinflammation and/or neurodegeneration in brain tissue of the animal subject, when compared to that of a control animal subject that received the same brain injury but is untreated with said candidate molecule.

In one embodiment, the animal subject receives lateral fluid percussion injury (LFPI). In one embodiment, LFPI is applied to a rat model. In a specific embodiment, the pressure pulse of the LFPI ranges from about 1.0 to 3.0 atm, 1.5 to 2.5 atm, or 2.0 to 2.2 atm.

### Therapeutic Compositions and Routes of Administration

The subject invention further provides therapeutic compositions that contain a therapeutically effective amount of the therapeutic agent of the subject invention and a pharmaceutically acceptable carrier or adjuvant. Particularly preferred pharmaceutical carriers for treatment of or amelioration of neuroinflammation in the central nervous system are carriers that can penetrate the blood/brain barrier.

The therapeutic agent used in the therapies can be in a variety of forms. These include for example, solid, semi-solid, and liquid dosage forms, such as tablets, pills, powders, liquid solutions or suspensions, suppositories, and injectable and infusible solutions. The preferred form depends on the intended mode of administration and therapeutic application.

In a preferred embodiment, the composition is formulated in accordance with routine procedures as a pharmaceutical composition adapted for local injection administration to human beings. Typically, compositions for local injection administration are solutions in sterile isotonic aqueous buffer. Generally, the ingredients are supplied either separately or mixed together in unit dosage form, for example, as a dry lyophilized powder or water free concentrate in a hermetically scaled container such as an ampoule or sachette indicating the quantity of active agent. Where the composition is administered by injection, an ampoule of sterile water for injection or saline can be provided so that the ingredients may be mixed prior to administration.

The subject invention also provides for a therapeutic method by administering therapeutic or pharmaceutical compositions in a form that can be combined with a pharmaceutically acceptable carrier. In this context, the compound may be, for example, isolated or substantially pure. The term “carrier” refers to a diluent, adjuvant, excipient, or vehicle with which the compound is administered. Such pharmaceutical carriers can be sterile liquids, such as water and oils, including those of petroleum oil such as mineral oil; vegetable oil such as peanut oil, soybean oil, and sesame oil; animal oil; or oil of synthetic origin.

Suitable carriers also include ethanol, dimethyl sulfoxide, glycerol, silica, alumina, starch, sorbitol, inosital, xylitol, D-xylose, manniol, powdered cellulose, microcrystalline cellulose, talc, colloidal silicon dioxide, calcium carbonate, magnesium carbonate, calcium phosphate, calcium aluminium silicate, aluminium hydroxide, sodium starch phosphate, lecithin, and equivalent carriers and diluents. Saline solutions and aqueous dextrose and glycerol solutions can also be employed as liquid carriers, particularly for injectable solutions.

Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The therapeutic composition, if desired, can also contain minor amounts of wetting or emulsifying agents, or pH buffering agents.

The amount of active ingredient that may be combined with the carrier materials to produce a single dosage form will vary, depending such as the type of the condition and the subject to be treated. The amount of active ingredient that may be combined with the carrier materials to produce a single dosage form will vary, depending such as the type of the condition and the subject to be treated. In general, a therapeutic composition contains from about 5% to about 95% active ingredient (w/w). More specifically, a therapeutic composition contains from about 20% (w/w) to about 80% or about 30% to about 70% active ingredient (w/w).

The compound of the subject invention can be formulated according to known methods for preparing pharmaceutically useful compositions. Formulations are described in detail in a number of sources which are well known and readily available to those skilled in the art. For example, Remington's Pharmaceutical Science by E. W. Martin describes formulations which can be used in connection with the subject invention. In general, the compositions of the subject invention will be formulated such that an effective amount of the bioactive compound(s) is combined with a suitable carrier in order to facilitate effective administration of the composition.

The therapeutic or pharmaceutical compositions of the subject invention can also be formulated as neutral or salt forms. Pharmaceutically acceptable salts include salts derived from hydrochloric, phosphoric, acetic, oxalic, or tartaric acids, etc., and salts derived from sodium, potassium, ammonium, calcium, ferric hydroxides, isopropylamine, triethylamine, 2-ethylamino ethanol, histidine, procaine, etc.

The compositions of the subject invention can be administered to the subject being treated by standard routes, including oral, inhalation, or parenteral administration including intravenous, subcutaneous, topical, transdermal, intradermal, transmucosal, intraperitoneal, intramuscular, intracapsular, intraorbital, intracardiac, transtracheal, subcutaneous, subcuticular, intraarticular, subcapsular, subarachnoid, intraspinal, epidural and intrasternal injection, infusion, and electroporation, as well as co-administration as a component of any medical device or object to be inserted (temporarily or permanently) into a subject.

The amount of the therapeutic or pharmaceutical composition of the subject invention effective in the treatment will depend on a variety of factors, such as the route of administration and the seriousness of the condition, and should be decided according to the judgment of the practitioner and each patient's circumstances. In general, the dosage ranges from about 0.01 μg/kg to about 10 mg/kg, about 0.01 μg/kg to about 1 mg/kg, about 0.01 μg/kg to about 100 μg/kg, about 0.01 μg/kg to about 10 μg/kg, or about 0.01 μg/kg to about 1 μg/kg. Such a unit dose may be administered once to several times (e.g. two, three and four times) every two weeks, every week, or every day.

In one embodiment, the compounds and compositions of the subject invention and any second therapeutic agent are administered simultaneously or sequentially to the patient, with the second therapeutic agent being administered before, after, or both before and after treatment with the compounds of the subject invention. Sequential administration may involve treatment with the second therapeutic agent on the same day (within 24 hours) of treatment with the subject compound. Sequential administration may also involve continued treatment with the second therapeutic agent on days that the subject compound is not administered.

In addition, in vitro assays may optionally be employed to help identify optimal dosage ranges. The precise dose to be employed in the formulation will also depend on the route of administration, and the seriousness of the disease, condition or disorder, and should be decided according to the judgment of the practitioner and each patient's circumstances. Effective doses may be extrapolated from dose-response curves derived from in vitro or animal model test systems.

### Materials and Methods

### Animals

All animal procedures were conducted in accordance with the NIH Guide for the Care and Use of Laboratory Animals and a protocol approved by the Institutional Animal Care and Use Committee at the University of South Florida. Male Sprague-Dawley rats (Harlan, Indianapolis, Ind.) weighing 250 to 300 g were housed in a climate-controlled room with water and laboratory chow available ad libidum. A total of 33 animals were used in this study.

### Induction of Lateral Fluid Percussion Injury (LFPI)

The LFPI model of TBI is an excellent model of clinical contusion without skull fracture [19, 20]. Animals receiving LFPI exhibit features of the primary TBI injury including the disruption of the blood brain barrier (BBB), the secondary injury, and diffuse axonal injury [21].

Animals were anesthetized using a mixture of ketamine/xylazine (ketamine 90 mg/kg, xylazine 10 mg/kg, Intraperitonial (IP). To deliver LFPI, a 1 mm diameter-craniotomy, centered at 2 mm lateral and 2.3 mm caudal to Bregma on the right side of the midline, was performed. A female luer-lock hub was implanted on the craniotomy site and secured with dental cement. The FPI device was then fastened to the luer-lock. All tubing was checked to ensure that no air bubbles had been introduced, after which a mild impact ranging from 2.0-2.2 atm was administered. Impact pressures were measured using a transducer attached to the point of impact on the fluid percussive device. The luer-lock was then detached. The craniotomy hole was sealed with bone wax and the scalp was sutured. Ketoprofen (5 mg/kg) was given to the rats to reduce postsurgical pain and inflammation. Rats were then placed back into their home cages and allowed to recover for 24 or 48 hours prior to subsequent experiments.

For all LFPI procedures, animals were excluded if either the impact did not register between 2.0 and 2.2 atm or if the dura was disturbed during the craniotomy prior to impact. In sham (control) animals craniotomy was performed at the same coordinates as the TBI animals but no impact was delivered. The craniotomy hole was sealed with bone wax, skin sutured and the animals were given Ketoprofen (5 mg/kg) before they were allowed to recover in their home cage for 24 or 48 hours. The animal was discarded from the experiment if the dura was broken during craniotomy.

### Tissue Collection

After 24 or 48 hours of LFPI, the animals were deeply anesthetized using ketamine and xylazine. Spleens were dissected out, and small pieces of the dissected spleens were collected in tube and immediately frozen on dry ice. Animals were then perfused with 0.9% saline followed by 4% paraformaldehyde in phosphate buffer (pH 7.4). The brains were harvested, post-fixed in paraformaldehyde, and saturated with increasing sucrose concentrations (20%, 30%) in phosphate-buffered saline (PBS, pH 7.4). Brains were then frozen, sectioned coronally at 30 μm thickness using a cryostat, thaw-mounted onto glass slides and stored at −20° C. prior to staining. For all staining experiments, three sections corresponding to 3.5, 4.5, and 5.5 mm caudal to Bregma were selected for analysis.

### Splenectomy

To remove the spleen from the anesthetized rat, a cranial-caudal incision was made lateral to the spine with the cranial terminus of the incision just behind the left rib cage. A small incision was made on the exposed muscle layer to access the spleen. The spleen was then pulled out through the incision, the splenic blood vessels were tied with 4.0 silk sutures and the spleen was removed by transecting the blood vessels distal to the ligature. The attached pancreatic tissues were detached from the spleen by blunt dissection and returned to the abdominal cavity before removal of the spleen. The muscle and skin incisions were sutured and the animals were allowed to survive for 24 or 48 hours.

RNA Extraction, Purification and cDNA Synthesis

Total RNA was extracted from 50 mg of frozen spleens using TRIZOL reagent (Invitrogen, Carlsbad, Calif.) according to manufacturer's instructions. Briefly, the samples were homogenized with 1 ml of TRIZOL reagent, incubated at room temperature for 5 minutes, and phase-separated by chloroform. Total RNA was precipitated by centrifugation with isopropyl alcohol. RNA was then purified using RNeasy mini kit (Qiagen, Valencia, Calif.) according to manufacturer's instructions.

RNA concentration and purity was determined with spectrophotometry at 260/280 nm and 260/230 nm. First strand cDNA was synthesized from the isolated RNA using the Superscipt III system (Invitrogen).

mRNA SuperArray Analysis

A panel of pro inflammatory cytokines and chemokines and their receptors was analyzed using SYBR green optimized primer assay (RT² Prolifer PCR Array) from SA bioscience (Frederick, Md.). Briefly, cDNA was synthesized from fresh frozen spleens as stated above. cDNA was mixed with the RT2 qPCR master mix and the mixture was aliquoted across the PCR array. The PCR was done in a CFX96 Real-Time C1000 thermcycler (BioRad) for 5 min at 65° C., 50 min at 50° C. and 5 min at 85° C. Control gene expression was normalized and target gene expression was expressed as fold increase or decrease compared to control. PCR data were analyzed using the SA Bioscience Excel program.

### Enzyme Linked Immunosorbent Assay (ELISA)

Spleen tissue lysate was prepared from 5 mg of fresh frozen tissue using protein lysis buffer containing NP-40. CCL20 was estimated by ELISA using DuoSet ELISA Development kit for CCL20 from R & D systems (Minneapolis, Minn.) according to manufacturer's instructions.

Briefly, 96 well sterile ELISA microplates were coated with anti-rat CCL20α antibody over night at room temperature. Next day, the plates were washed and blocked with bovine serum albumin (BSA). Plates were incubated sequentially with standards or samples for 2 hours, the detection antibody (biotinylated goat anti-rat CCL20a antibody) for 2 hours, streptavidin-HRP for 20 minutes, and substrate solution (1:1 mixture of H2O2 and tetramethylbenzidine) for 20 minutes; the reactions were stopped with 2N H₂SO₄.

All the incubations were performed at room temperature. Each incubation was separated by thorough wash of the microplate with wash buffer. The optical density of each well was determined at 450 nm using Synergy H4 Hybrid reader from BioTek.

Total protein concentrations from the same samples were determined using the BCA protein assay method. CCL20 was expressed as pg per μg of total protein present in the tissue.

### Fluor-Jade Histochemistry

Fluoro-Jade (Histochem, Jefferson, Ark.) staining was performed to label degenerating neurons. This method was adapted from that originally developed by Schmued et al. 1221.

Briefly, thaw-mounted sections were placed in 100% ethanol for 3 min followed by 70% ethanol and deionized water for 1 min each. Sections were then oxidized using 0.06% KMnO4 solution for 15 min followed by three rinses in ddH2O for 1 min each. Sections were then stained in 0.001% solution of Fluoro-Jade in 0.1% acetic acid for 30 min. Slides were again rinsed, dried at 45° C. for 20 min, cleared with xylene, and coverslipped using DPX mounting medium (Electron Microscopy Sciences, Ft. Washington, Pa.).

### Isolectin IB4 Staining

Brain sections were washed with modified PBS (PBS with 0.5 mM CaCl₂, pH 7.2) and permeabilized with buffer containing 10% goat serum, 3% lysine, 0.3% triton X-100 in modified PBS for 1 hour at room temperature. Sections were then incubated over night at 4° C. with 5 μg/ml Alexa 488 conjugated Isolectin IB4 (Molecular Probes) dissolved in modified PBS with 0.3% triton x-100 and 2% goat serum. Sections were then washed with modified PBS, mounted with Vecta-Shield mounting medium with DAPI, and viewed using the FITC filter of an Olympus Fluorescent microscope. Images were taken using Olympus DP70 imaging system and IB4 positive cells were quantified using Image J quantification program.

### Immunohistochemistry

Spleen, thymus or brain tissue sections were washed with PBS for 5 min. incubated in 3% hydrogen peroxide for 20 min and washed 3 times in PBS. They were then heated in antigen unmasking solution (1:100; Vector Laboratories Inc., Burlingame, Calif.) for 20 min at 90° C., incubated for 1 h in permeabilization buffer (10% goat serum, 0.1% Triton X-100 in PBS) and incubated overnight at 4° C. with either rabbit anti-CCL20 primary anti-body (1:1000) or mouse monoclonal anti-CD11b antibody (1:400) (Abeam, Cambridge, Mass.) in antibody solution (5% goat serum, 0.05% Triton X-100 in PBS).

The following day, sections were washed with PBS and incubated 1 h at room temperature with secondary antibody (biotinylated goat anti-rabbit, 1:400, Vector Laboratories Inc., Burlingame, Ca or Alexafluor 594 conjugated anti-mouse antibody, 1:50 or DyLight 594 conjugated anti-rabbit antibody, 1:50) in antibody solution.

Sections incubated with biotinylated anti-rabbit antibody were then washed in PBS, incubated in avidin-biotin complex mixture (ABC, 1:100; Vector Laboratories Inc, Burlingame, Ca) for 1 h, washed again and visualized using DAB/peroxide solution (Vector Laboratories Inc). After three washes, sections were dried, dehydrated with increasing concentrations of ethanol (70%, 95%, 100%), cleared with xylene and cover-slipped with Vectamount mounting medium. Sections incubated with mouse anti-CD11b antibody followed by alexafluor 594-conjugated anti-mouse antibody were washed three times with PBS and used for double staining with IB4. Some of the anti-CCL20 antibodies followed by DyLight 594-conjugated anti-rabbit antibody treated sections were incubated with Alexa fluor 488-conjugated mouse anti-neuronal nuclei (NeuN) monoclonal antibody (1:100; Millipore, Temecula, Calif.) 3 hours at room temperature, washed with PBS, dried and cover slipped with vectamount mounting medium with DAPI.

For peroxidase detection, brain tissue sections were washed with PBS for 5 min and incubated in 3% hydrogen peroxide for 20 min. Sections were then washed 3 times in PBS, incubated for 1 hour in permeabilization buffer (2% serum, 0.3% Triton X-100 and 0.3% IM lysine in PBS), and incubated overnight at 4° C. with rabbit anti-mouse CCL20α primary antibody (Abeam, Cambridge, Mass.) in antibody solution (2% goat serum, 0.3% Triton X-100 in PBS). The following day, sections were washed with PBS and incubated for 1 hour at room temperature with secondary antibody in antibody solution. Sections were then washed in PBS, incubated in Avidin-Biotin Complex (ABC; Vector Laboratories Inc, Burlingame, Ca) mixture for 1 hr, washed again and visualized using a DAB/peroxide solution (Vector Laboratories Inc). After 3 final washes, sections were dried, dehydrated with increasing concentrations of EtOH (70%, 95%, 100%), and cleared with xylene and cover-slipped with DPX.

### TUNEL Staining

The nuclear DNA fragmentation, an important marker for apoptotic cells, was measured using DeadEnd Fluorimetric TUNEL system (Promega, Madison, Wis.) according to the manufacturer's instruction. Briefly, 4% PFA fixed 30μ thick cryosections were permeabilized with 20 μg/ml Proteinase K solution at room temperature for 8 minutes, followed by 4% PFA in PBS for 5 minutes.

The sections were then washed in PBS and equilibrated with the equilibration buffer (200 mM potassium cacodylate, pH 6.6; 25 mM Tris-HCl, pH 6.6; 0.2 mM DTT; 0.25 mg/ml BSA and 2.5 cobalt chloride) for 10 minutes at room temperature. The sections were then incubated at 37° C. for 1 hour with incubation buffer containing equilibration buffer, Nucleotide mix and rTdT enzyme mix, covered with plastic cover slip to avoid exposure to light.

The cover slips were removed carefully and the reactions were stopped with 2×SSC. The sections were then washed with PBS and mounted with VectaShield mounting medium with DAPI.

The green fluorescence of fluorescein-12-dUTP was detected in the blue background of DAPI under fluorescence microscope. Images were taken and apoptotic nuclei were quantified using the image J quantification program.

### Image Analysis and Quantification

All quantitation was performed using the NIH Image J software. For immunohistochemical analysis, images were acquired using a Zeiss Axioskop2 controlled by Openlab software (Improvision Ltd., Lexington, Mass.). Photomicrographs were captured at 20× magnification with a Zeiss Axiocam Color camera. All images were captured at the same exposure and digital gain settings to minimize confounds of differential background intensity or false-positive immunoreactivity across sections.

The channels of the RGB images were split and the green channel was used for quantitation of the FJ, IB4 and TUNEL staining images. The CCL20 images were converted to gray-scale before quantitation. The single channel or gray-scale images were then adjusted for brightness and contrast to exclude noise pixels. The images were also adjusted for the threshold to highlight all the positive cells to be counted and a binary version of the image was created with pixel intensities 0 and 255. Particle size was adjusted to exclude the small noise pixels from the count. Circularity was adjusted to between 0 and 1 to discard any cell fragments, processes or tissue aggregates resulting in false labelling from the quantitation. The same specifications were used for all sections. Cell counts of sections from 3.5, 4.5 and 5.5 mm caudal to the bregma were summed to represent the number of positive cells from each brain. The results for the FJ, TUNEL, IB4 and CCL20 immunoreactivity were expressed as mean number of positive cells±S.E.M. CCL20 immunoreactivity of the thymus or the spleen was expressed as mean area of immunoreactivity±S.E.M.

Fluoro-Jade-stained tissue sections were photographed at 1.25× magnification with an Olympus IX71 microscope controlled by DP manager software (Olympus America Inc., Melville, N.Y.). Images were then edited with Jase Paintshop Pro to sharpen and enhance contrast to the same specifications across sections. Total area of neurodegeneration, as indicated by Fluoro-Jade staining, was measured using NIH Image J software.

For IB4 and TUNEL assay quantifications, images were captured at 20× magnification. Only the green channel of each image was analyzed for quantification. The images were adjusted for brightness and contrast. The threshold and the circularity were adjusted to discard any false labelling from the quantification. The results were expressed as mean number of positive cells±S.E.M.

Mixed Glial Cultures and oligodendrocytes Culture Purification

Mixed glial cultures were prepared from postnatal day 2 rats and oligodendrocytes (OL) cultures were purified using the shaking and differential adherence method as previously described (26). Cell preparations were seeded (1.5×10⁷) into flasks, OLs were purified from these preparations after 8 DIV and plated onto glass poly-l-lysine-treated coverslips. Following a 7 day proliferation period, the PDGF-AA was withdrawn for 5 days to induce OL differentiation into the mature phenotype. Experiments were conducted within three weeks.

### Primary Neuronal Cultures

Cortices from E18 rat embryos were dissociated with a solution of 0.25% trypsin/2.21 mM EDTA for 10 min at 37° C. The solution was triturated to obtain a uniform single cell suspension. After centrifugation, the supernatant was aspirated off and the cells were re-suspended in DMEM (Mediatech, Mannasas, Va.). Trypan blue exclusion was used to count viable cells and 3×10⁵ cells in a final volume of 1 mL were seeded on 24 well poly-L-lysine treated culture plates. After 24 h, the medium was changed to neurobasal complete (neurobasal medium (Invitrogen), B-27 (Invitrogen), 0.05 mM L-glutamine (Mediatech) and cells were cultured for 7 days. Cells were used for oxygen glucose deprivation (OGD) experiments following one medium change, as determined by experimental grouping.

### Oxygen Glucose Deprivation

Primary cells previously seeded onto glass coverslips were randomly assigned to one of five conditions: OGD (DMEM without glucose)+vehicle, OGD+CCL20 (200 ng; Rad Systems, Minneapolis, Minn.), normoxia (DMEM with glucose) only, normoxia+vehicle, or normoxia+CCL20. Cells undergoing OGD were placed in an airtight hypoxic chamber. The chamber was then flushed with hypoxic gas (95% N₂, 4% CO₂, 1% O₂; Airgas, Tampa, Fla.) for 15 min and scaled for the duration of exposure. Normoxic cells were maintained in a standard tissue culture incubator. Cultures were subjected to OGD or normoxia for 24 h at 37° C. The medium from each well was collected, clarified by centrifugation, and lactate dehydrogenase (LDH) analyzed immediately.

### Lactate Dehydrogenase Assay

Cell death in culture was determined using the lactate dehydrogenase (LDH) assay (Takara Bio, Inc., Madison, Wis.). Briefly, 100 μl of tissue culture medium from each experimental group was added to a 96-well plate and 100 μl of LDH reagent was added to each well. Plates were incubated for 30 min at 25° C. and absorbances were read on a microplate reader at a 548 nm wavelength. The absorbance of medium only wells was subtracted from the total absorbance of each experimental treatment group to control for background LDH activity.

### Statistical Analysis

All data are presented as mean±S.E.M. One-way ANOVA with Bonferroni's post test was used to determine the level of statistical significance between groups. A p value less than 0.05 was considered statistically significant.

### EXAMPLES

Following are examples that illustrate embodiments and procedures for practicing the invention. These examples should not be construed as limiting. All percentages are by weight and all solvent mixture proportions are by volume unless otherwise noted.

### Example 1

### Regional Distribution of Neurodegeneration after TBI

To date, the assessment of TBI injury has been inconsistent across the laboratories. In addition, there is a lack of reliable, quantitative approaches for assessing neural injury. These have impeded efforts to develop novel treatments for TBI.

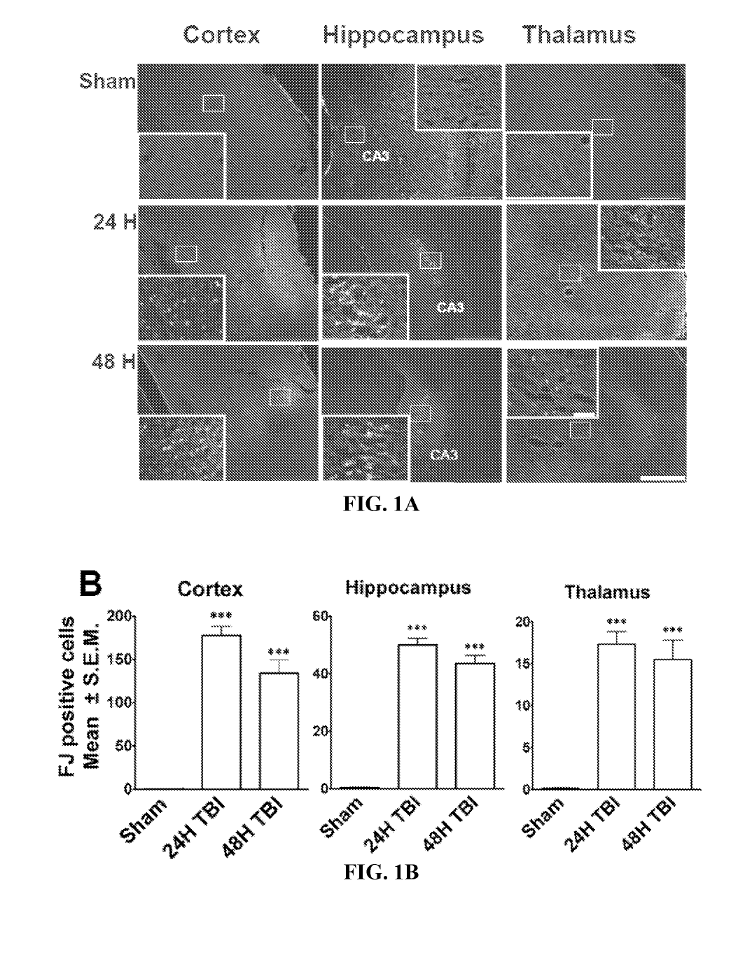

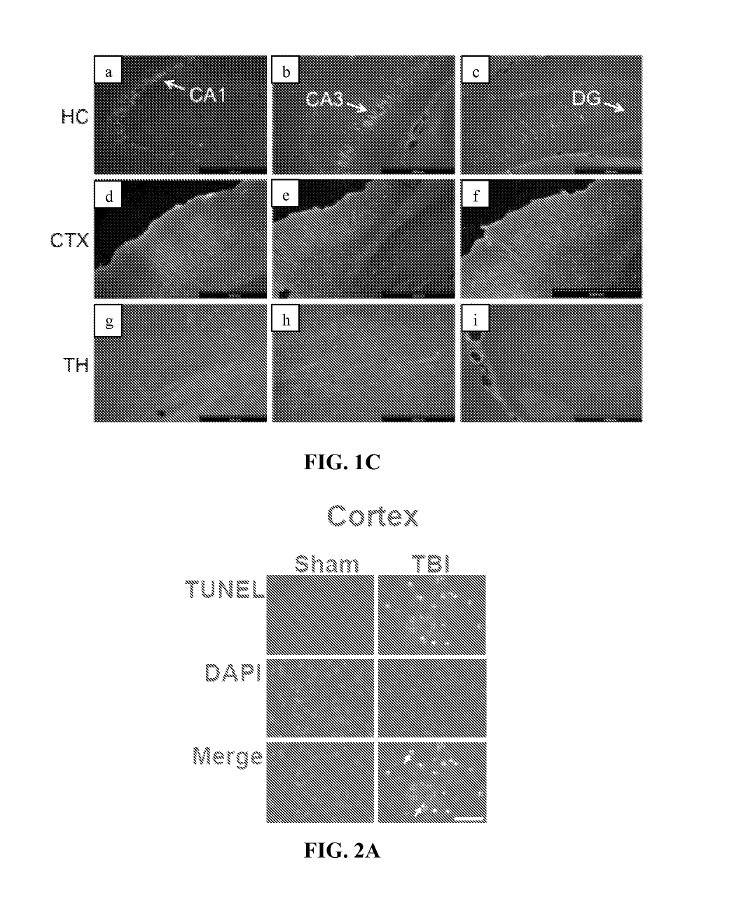

This Example conducts a detailed investigation throughout the brain to determine which regions of the brain exhibit consistent, prominent neurodegeneration. Briefly, rats were subjected to mild LFPI, and the brains were sectioned and stained with Fluoro-Jade. FIG. 1 shows a consistent profile, where the majority of Fluoro-Jade-positive cells were found within the cortex, hippocampus and thalamus. Cortical Fluoro-Jade was ubiquitous and was present at various levels throughout the brain. Hippocampal FJ staining was localized to the pyramidal cell layers (FIG. 1), while some diffuse labelling throughout the general structure was also evident. The thalamic staining was diffuse and sparsely distributed. Quantitation revealed that the neurodegeneration in these regions significantly increased at both 24 and 48 h post-impact relative to sham-operated controls.

Additionally, the data showed that Fluoro-Jade-stained degenerating hippocampal neurons were restricted to the ipsilateral hemisphere, whereas cortical and thalamic labelling was also detected in the contralateral hemisphere. Based upon these data, neuronal injury assessment in subsequent experiments was limited to the hippocampal CA1 and CA3 pyramidal cell layers, as those cell layers showed highly reproducible injury and are known to mediate cognitive functions impaired by TBI injury.

### Example 2

### Mild TBI Induced Internucleosomal DNA Fragmentation in the Cortex and Hippocampus

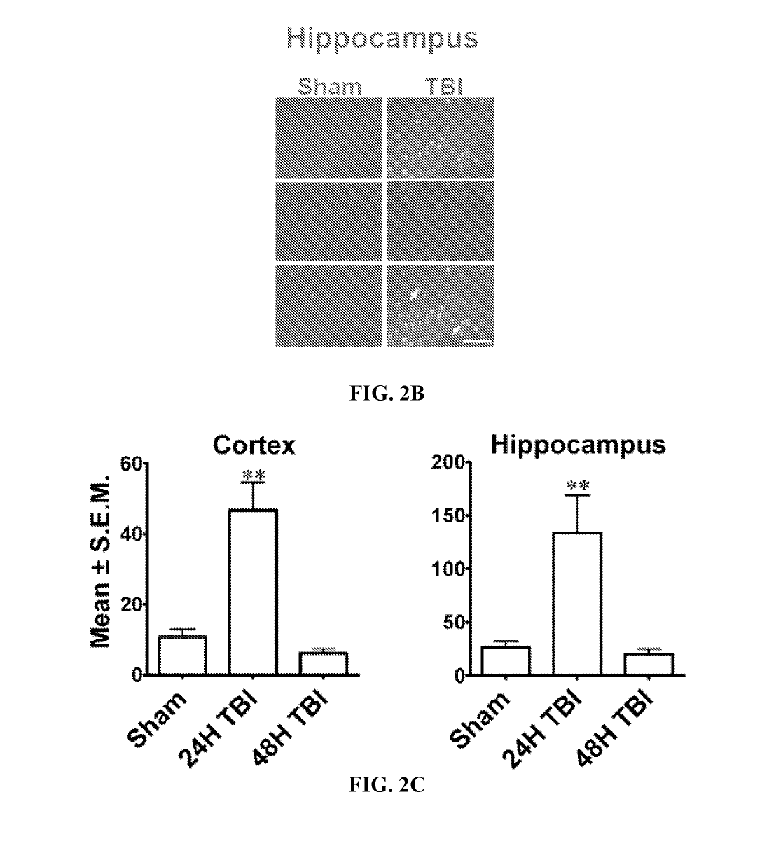

Internucleosomal DNA fragmentation, an important marker for apoptotic cells, was assessed by the Terminal Deoxynucleotidyl Transferase Biotin-dUTP Nick End Labeling (TUNEL) histochemistry. Few TUNEL-positive cells were detected in the contralateral hemisphere. While the ipsilateral thalamus showed sparse TUNEL staining in some sections, this was not a consistent finding throughout the experiment. The majority of TUNEL-stained nuclei were detected at 24 h post-TBI in the ipsilateral cortex (FIG. 2A) and hippocampus (FIG. 2B), while sections from sham-operated controls were predominantly devoid of TUNEL staining in these regions (FIG. 2A-2B) and showed only background levels of fluorescence. By 48 h after TBI, sections showed very few TUNEL-positive cells in the cortex and hippocampus and resembled sham-operated controls. Quantitation revealed a significant increase in TUNEL-positive cells in both cortex and hippocampus 24 h post TBI as compared to sham-operated control groups (FIG. 2C).

### Example 3

### Activation of Microglia in the Brain Following Mild TBI

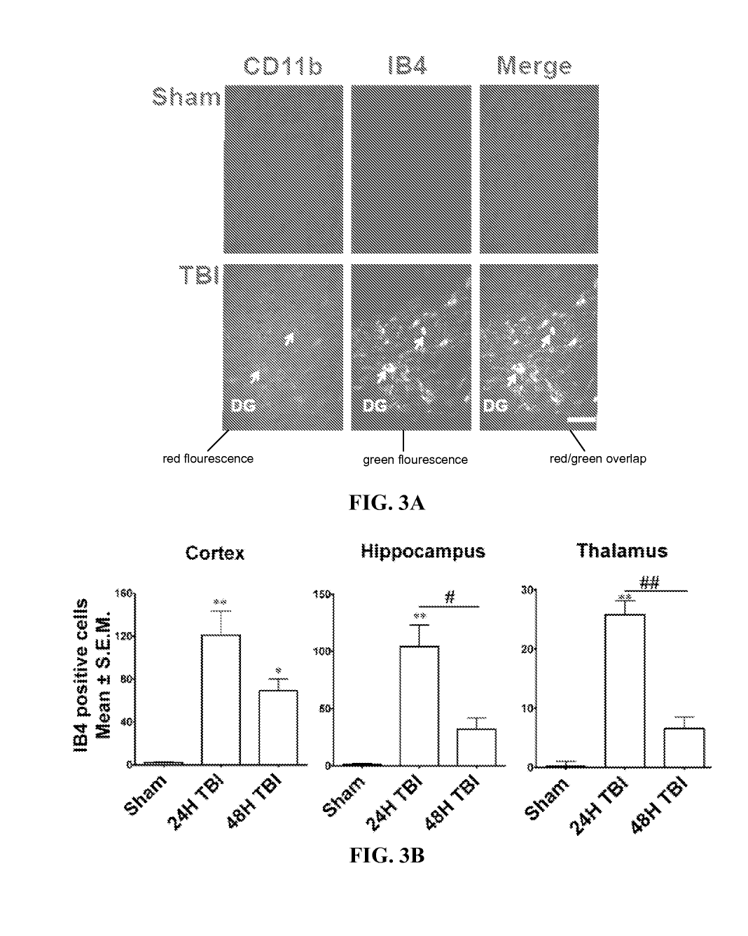

Isolectin-IB4, a 114 kD protein isolated from the seeds of the African legume—Griffonia simplicifolia, has been shown to have a strong affinity for brain microglial cells. To elucidate the local inflammatory response following mild TBI, Alexafluor 488 conjugated IB4 was used to label activated microglia in the rat brains (FIG. 3). While IB4 labelling was primarily restricted to the ipsilateral hemisphere, sparse labelling was detected within the contralateral hippocampus (data not shown). IB4-positive cells were abundant in the hippocampus, especially in the dentate gyrus (FIG. 3A). Microglia cells were also found in the cortex and thalamus following TBI.

CD11b, an activated microglial marker, was also found in the cells of the cortex and hippocampus (dentate gyrus, FIG. 3A) of the ipsilateral side. Confocal microscopy revealed that most but not all IB4⁺ cells in the cortex or hippocampus were also CD11b⁺ (FIG. 3A). Quantitation showed that the number of IB4-positive cells was significantly increased in each of these brain regions 24 h after TBI, while number of IB4⁺ cells in these regions 48 h post-TBI did not significantly differ from sham-operated controls (FIG. 3B). These observations indicate that an inflammatory response was mounted within the brain parenchyma as early as 24 h after the injury involving microglial activation/migration to the site of injury.

### Example 4

### Identification of CCL20 as the Major Inflammatory Gene Expressed in the Spleen and Thymus Following TBI

It is suggested that, in addition to local inflammatory response, the activation of systemic inflammatory response is critical for inducing TBI-associated neuropathies. Although a number of cytokines and chemokines have been studied, no key systemic inflammatory molecule has been identified.

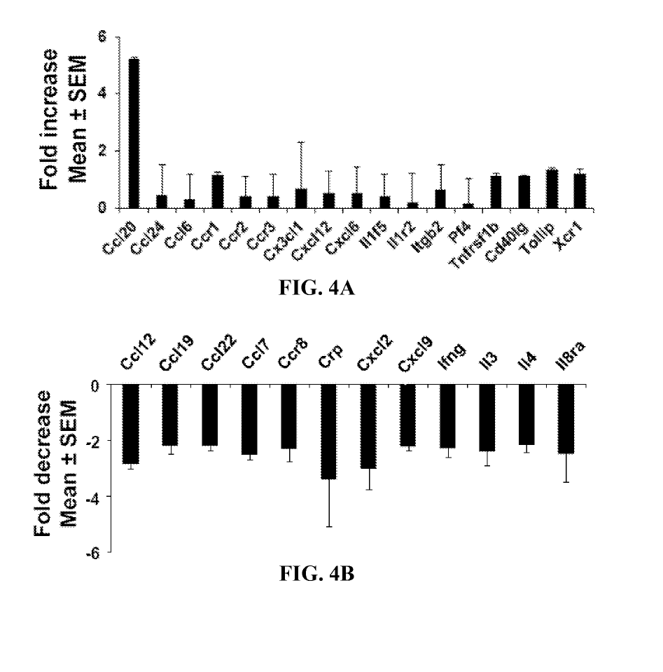

The spleen is involved in the systemic inflammatory response in various injury models. In this Example, a comprehensive SuperArray analysis was performed on spleen RNA from two separate experiments, to identify alterations in the expression of 84 genes associated with pro-inflammatory signaling after LFPI (FIG. 4). The SuperArray data show that most genes were down-regulated 24 hours and/or 48 hours after LFPI. Among the genes upregulated after LFPI, CCL20 was upregulated by five-fold compared to controls (FIG. 4A) 24 hours and/or 48 hours after LFPI. The results reveal that CCL20 is a pro-inflammatory, systemic marker for TBI.

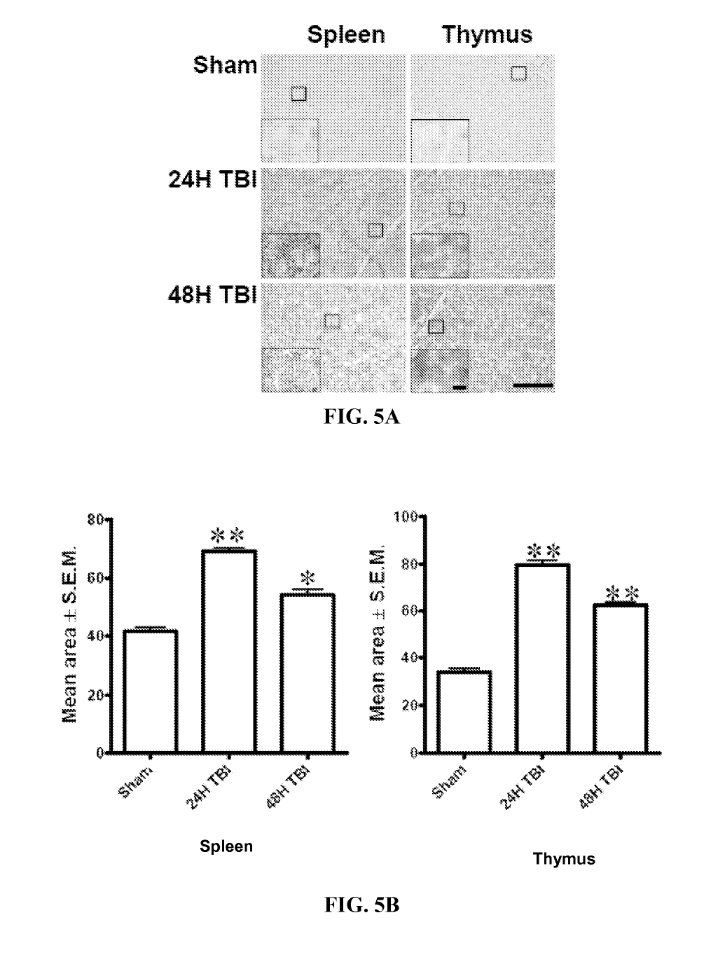

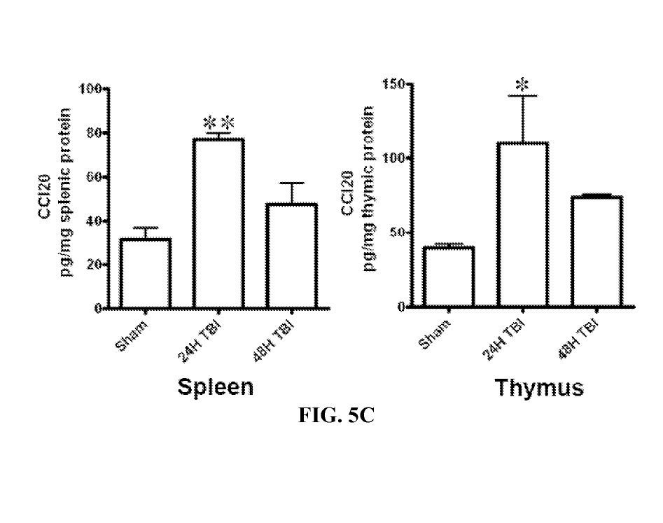

To determine whether alterations in CCL20 mRNA paralleled protein expression, ELISAs and immunohistochemistry were performed on spleen tissues. Immunohistochemistry on spleen tissues indicated significant up-regulation of CCL20 expression at 24 h after TBI, as indicated by the increase in mean area of CCL20 intensity. Significant expression of the CCL20 protein was also observed 48 h after impact (FIGS. 5A-B).

The immunohistochemical observation was also supported by the results obtained from ELISA of spleen tissues. The results show at least two-fold up-regulation of CCL20 protein expression 24 h after TBI (FIG. 5C).

In addition to spleen, the thymus also expressed CCL20 at 24 h after TBI as evident from the immunohistochemical labelling of thymus (FIGS. 5A and 5B) and ELISA for CCL20 of thymic tissues (FIG. 5C). The results show that CCL20 chemokine signaling plays a role in the systemic inflammatory response, and that the spleen and thymus respond as early as 24 h after TBI.

### Example 5

### Elevated CCL20 Expression in the Brain Following TBI-Induced Neurodegeneration

Data from the regional injury distribution experiments showed that mild TBI resulted in highly reproducible cellular injury within the cortex as well as the hippocampus. Because splenic CCL20 expression was increased in the acute phase of TBI injury (24 h post-insult) and the splenic inflammatory response is known to exacerbate neural injury, experiments were performed to determine whether CCL20 expression is associated with neural injury.

Brain sections from animals subjected to mild TBI or sham-TBI were immunostained for CCL20 expression using an antibody generated against the same CCL20 antigen that was used to immunostain the spleen and thymus sections (FIG. 6).

CCL20 immunoreactivity was observed in the cortex and hippocampus 48 h after TBI. In the cortex, CCL20 was expressed in the ipsilateral as well as contralateral sides. The immunoreactivity was observed in the CA1 and CA3 hippocampal pyramidal cell layers and was restricted to ipsilateral side of the brain. CCL20 immunoreactivity was absent in the 24 h group.

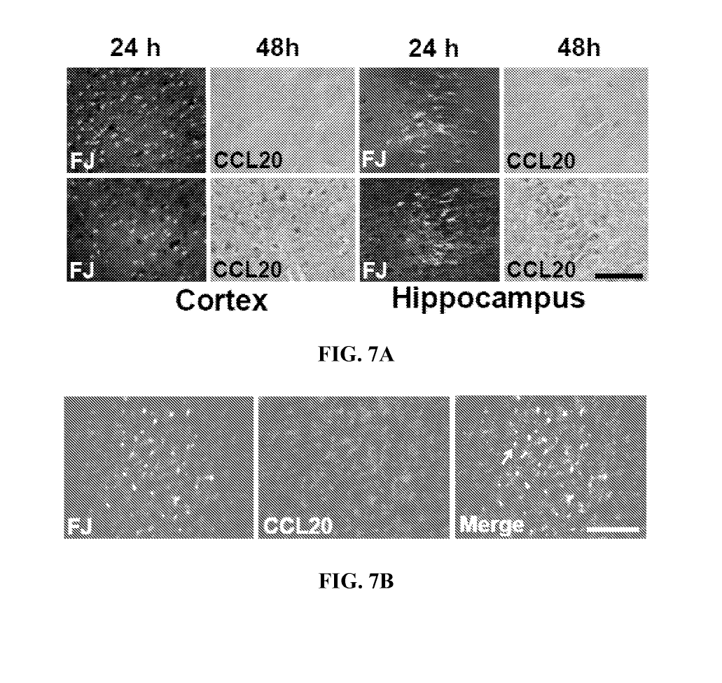

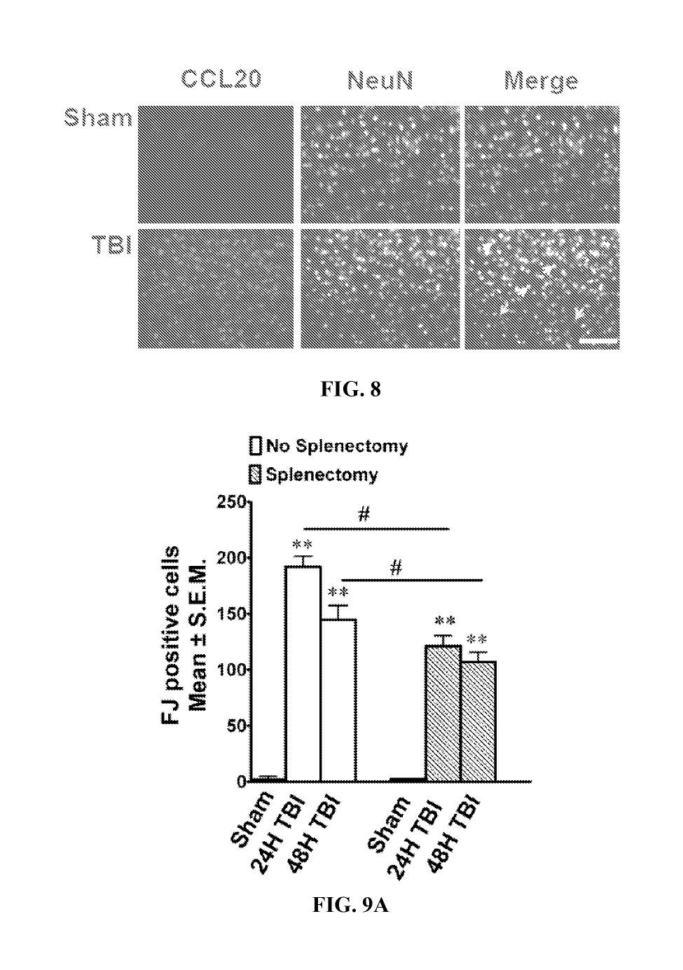

Additionally, CCL20-positive neuronal cell bodies displayed pyknotic morphology and were surrounded by areas devoid of tissue (FIGS. 6A and 7A). The immunohistochemical observation was further supported by the quantitation of the CCL20-positive cell bodies which showed a significant increase in CCL20-positive neurons in the cortex and hippocampus of rats euthanized 48 h post-TBI compared to 24 h or sham control rats (FIG. 6B). Although CCL20 immunoreactivity was not seen in the damaged neurons at 24 h, it was expressed by the neurons of cortex and hippocampus (FIG. 7A), including the degenerating ones in these regions, at 48 h after impact as evident by the co-localization of FJ and CCL20 stainings (FIG. 7B). CCL20 expressing cells in the cortex (FIG. 8) and hippocampus were mostly neurons as they were also NeuN positive.

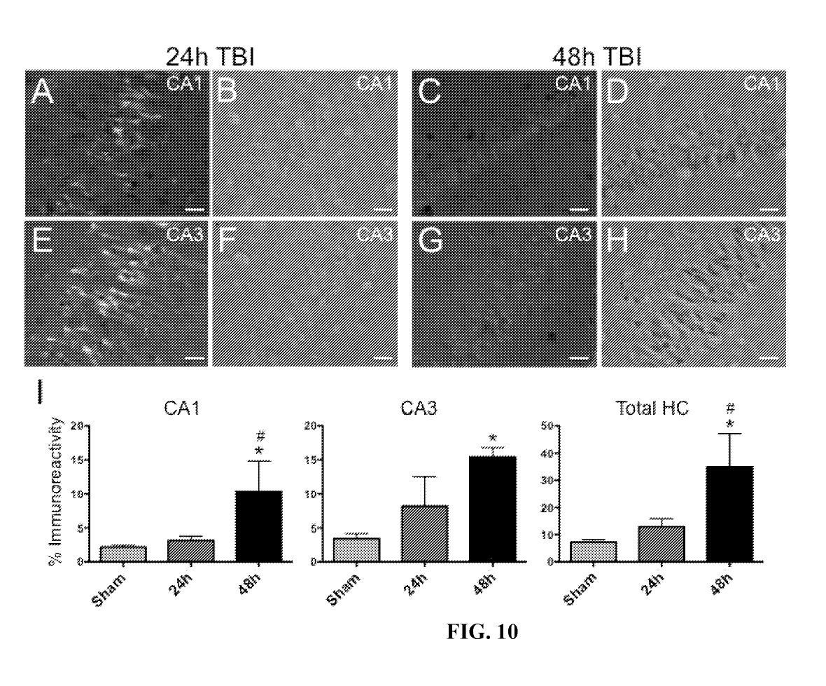

In a separate set of experiments, moderate TBI resulted in highly reproducible cellular injury within the CA1 and CA3 hippocampal pyramidal cell layers. Briefly, animals subjected to TBI or sham-TBI were euthanized at 24 or 48 h post-insult, and brains were sectioned for histological assessment of neurodegeneration, as measured by Fluoro-Jade staining, and CCL20 expression using an antibody generated against the CCL20 antigen (FIG. 10).

The results show that TBI produced cellular injury that was localized to the CA1 (FIG. 10A) and CA3 (FIG. 10E) hippocampal pyramidal cell layers at 24 h post-insult. Adjacent sections showed no CCL20 immunoreactivity in hippocampal neurons (FIG. 10B,F). Sections from rats euthanized 48 h post-TBI showed very little Fluoro-Jade staining (FIG. 10C,G), indicating that the acute phase of neurodegenerative injury does not persist to 48 h after the insult. CCL20 immunoreactivity was abundant throughout the CA1 (FIG. 10D) and CA3 (FIG. 10H) pyramidal cell layers 48 h post-TBI, and neuronal cell bodies displaying pyknotic morphology were surrounded by areas devoid of tissue. Quantification revealed significant (p<0.05) elevations in CCL20 in both the CA1 and CA3 hippocampal pyramidal cell layers (FIG. 10I).

The results show that CCL20 expression is increased in the brain due to TBI-induced neuronal injury at a later time point than the systemic increase of the same chemokine in response to mild TBI. The results also show that CCL20 plays a role in the neural injury and inflammatory reaction in the brain.

### Example 6

### Attenuation of TBI-Induced Neurodegeneration and CCL20 Expression in the Cortex by Splenectomy

To evaluate the significance of the spleen in LFPI-induced neurodegeneration, splenectomy was performed immediately after the induction of TBI. FJ histochemistry and CCL20 immunostaining were performed to evaluate the extent of damage in splenectomised animals.

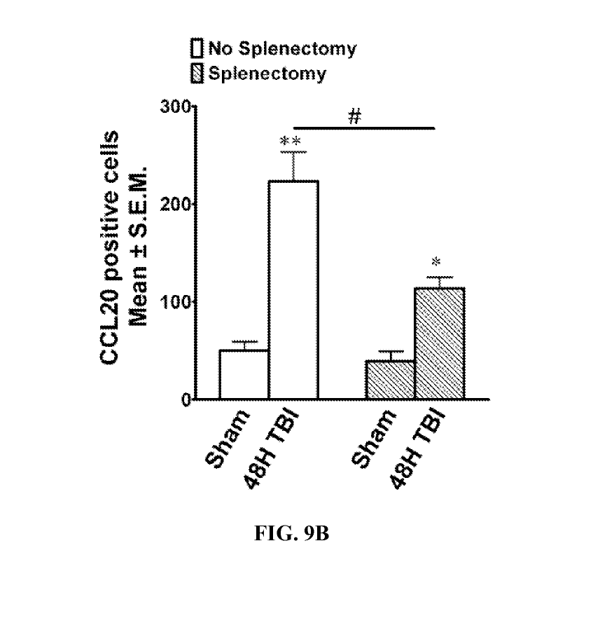

The results show that in splenectomised rats, the number of FJ-positive cells was significantly reduced, when compared to non-splenectomised animals at the same time points; while within the splenectomy group, the number of FJ-positive cells was significantly increased after TBI, when compared to splenectomised shams (FIG. 9A). Splenectomy also reduced CCL20 expression in the cortex 48 h after TBI. In splenectomised rats, CCL20 expression increased significantly, when compared to splenectomised sham animal; however, the CCL20 expression was reduced significantly when the spenectomised TBI rats were compared to the non-splenectomised TBI group. The results show that the spleen plays a role in TBI-induced neurodegeneration and CCL20 expression in the rat brain after mild TBI.

### Example 7

### Upregulation of CCL20 in White Matter after LFPI

As TBI produces injury to both gray and white matter, immunohistochemistry was performed to determine whether CCL20 is up-regulated in the white matter rich region of the external capsule following LFPI.

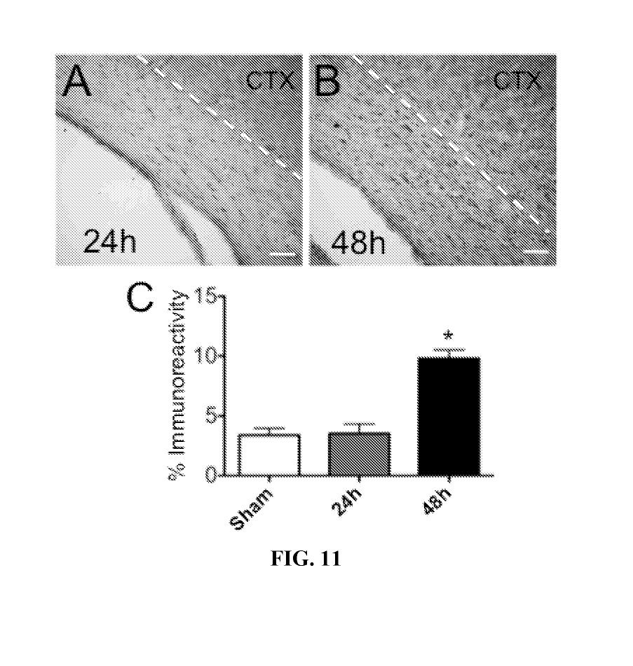

The results show that CCL20 expression was predominantly localized to cell bodies, although it occasionally appeared to label processes. Tissues from animals euthanized 24 h after TBI (FIG. 11A) showed few CCL20-positive cells, displayed faint immunoreactivity when it was present, and staining resembled that of sham rats. By 48 h post-LFPI, CCL20 immunoreactivity was prevalent throughout the external capsule (FIG. 11B) and cells labeled more intensely compared to 24 h TBI and sham-TBI rats. Quantification (FIG. 11C) showed that CCL20 expression was significantly elevated 48 h post-LFPI relative to 24 h and sham-operated controls (p<0.05).

### Example 8

### CCL20 is Toxic to Cultured Neurons and Oligodendrocytes Exposed to Oxygen Glucose Deprivation

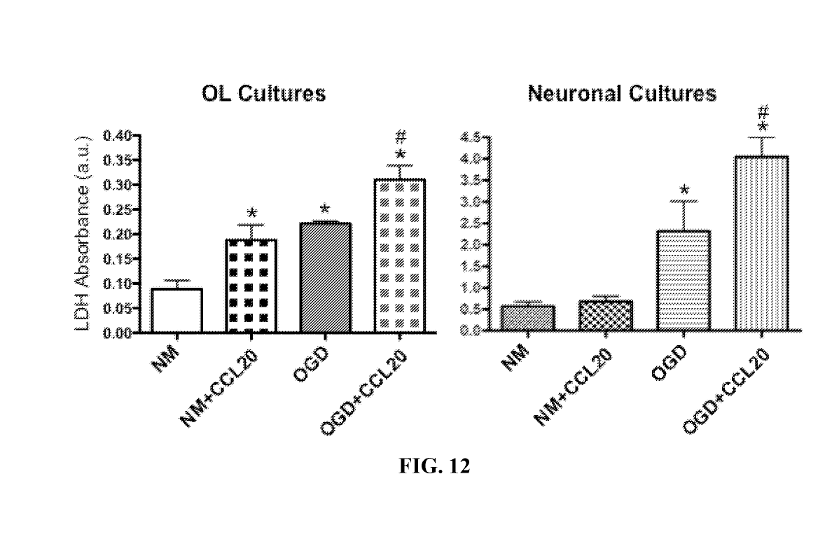

As CCL20 was elevated in neurons and white matter at time points consistent with neurodegenerative injury, experiments were performed using cultured primary neurons and oligodendrocytes (OLs) to determine whether this chemokine promotes cellular toxicity during periods of oxygen glucose deprivation (OGD) (FIG. 12).

Briefly, rat primary neurons or OLs were exposed to normoxia or OGD in the presence of 200 ng recombinant CCL20 or vehicle. Following the exposure, culture medium was collected and the LDH assay was performed to assess cellular death.

The results show that, OGD significantly increased (p<0.05) OL and neuronal cell death in the presence and absence of CCL20 compared to normoxia. Application of CCL20 significantly increased (p<0.05) OL and neuronal cell death relative to cells exposed to OGD in the absence of this chemokine. Additionally, CCL20 elicited toxicity to OLs under normoxic conditions but did not affect neuronal viability in the absence of OGD.

All patents, patent applications, provisional applications, and publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### REFERENCES

1. Fabrizio, K. S. and N. L. Keltner, Traumatic brain injury in operation enduring freedom/operation iraqi freedom: a primer. Nurs Clin North Am, 2010. 45(4): p. 569-80.
2. Galarneau, M. R., et al., Traumatic brain injury during Operation Iraqi Freedom: findings from the United States Navy-Marine Corps Combat Trauma Registry. J Neurosurg, 2008. 108(5): p. 950-7.
3. Macgregor, A. J., A. L. Dougherty, and M. R. Galarneau, Injury-Specific Correlates of Combat-Related Traumatic Brain Injury in Operation Iraqi Freedom. J Head Trauma Rehabil, 2010.
4. MacGregor, A. J., et al., Prevalence and psychological correlates of traumatic brain injury in operation iraqi freedom. J Head Trauma Rehabil, 2010. 25(1): p. 1-8.
5. Fernaud-Espinosa, I., M. Nieto-Sampedro, and P. Bovolenta, Differential activation of microglia and astrocytes in aniso-and isomorphic gliotic tissue. Glia, 1993. 8(4); p. 277-91.
6. Reid, D. M., et al., Mitosis and apoptosis of microglia in vivo induced by an anti-CR3 antibody which crosses the blood-brain barrier. Neuroscience, 1993. 56(3): p. 529-33.
7. Ghirnikar, R. S., Y. L. Lee, and L. F. Eng, Inflammation in traumatic brain injury: role of cytokines and chemokines. Neurochem Res, 1998. 23(3): p. 329-40.
8. Utagawa. A., et al., Systemic inflammation exacerbates behavioral and histopathological consequences of isolated traumatic brain injury in rats. Exp Neurol, 2008. 211(1): p. 283-91.
9. Morganti-Kossmann, M. C., et al., Modulation of immune response by head injury. Injury, 2007. 38(12): p. 1392-400.
10. Schutyser, E., S. Struyf, and J. Van Damme, The CC chemokine CCL20 and its receptor CCR6. Cytokine Growth Factor Rev, 2003. 14(5): p. 409-26.
11. Ohta, H., et al., Therapeutic time window of post-ischemic mild hypothermia and the gene expression associated with the neuroprotection in rat focal cerebral ischemia. Neurosci Res, 2007. 57(3): p. 424-33.
12. Dicu-Nosjean, M. C., et al., Macrophage inflammatory protein 3alpha is expressed at inflamed epithelial surfaces and is the most potent chemokine known in attracting Langerhans cell precursors. J Exp Med, 2000. 192(5): p. 705-18.
13. Chabaud, M., G. Page, and P. Miossec, Enhancing effect of IL-1, IL-17, and TNF-alpha on macrophage inflammatory protein-3alpha production in rheumatoid arthritis: regulation by soluble receptors and Th2 cytokines. J mmunol. 2001. 167(10): p. 6015-20.
14. Matsui, T., et al., Selective recruitment of CCR6-expressing cells by increased production of MIP-3 alpha in rheumatoid arthritis. Clin Exp Immunol. 2001. 125(1): p. 155-61.
15. Helmy, A., et al., The cytokine response to human traumatic brain injury: temporal profiles and evidence for cerebral parenchymal production. J Cereb Blood Flow Metab, 2010.
16. Comerford, I., et al., An immune paradox: How can the same chemokine axis regulate both immune tolerance and activation?: cCR6/CCL20: A chemokine axis balancing immunological tolerance and inflammation in autoimmune disease. Bioessays, 2010.
17. McKimmie, C. S. and G. J. Graham, Astrocytes modulate the chemokine network in a pathogen-specific manner. Biochem Biophys Res Commun, 2010. 394(4): p. 1006-11.
18. Reboldi, A., et al., C-C chemokine receptor 6-regulated entry of TH-17 cells into the CNS through the choroid plexus is required for the initiation of EAE. Nat Immunol, 2009. 10(5); p. 514-23.
19. Thompson, H. J., et al., Lateral fluid percussion brain injury: a 15-year review and evaluation. J Neurotrauma, 2005. 22(1): p. 42-75.
20. Cernak, I., Animal models of head trauma. NeuroRx, 2005. 2(3): p. 410-22.
21. Cernak, I., et al., The pathobiology of moderate diffuse traumatic brain injury as identified using a new experimental model of injury in rats. Neurobiol Dis, 2004. 17(1): p. 29-43.
22. Schmued, L. C., C. Albertson, and W. Slikker, Jr., Fluoro-Jade: a novel fluorochrome for the sensitive and reliable histochemical localization of neuronal degeneration. Brain Res, 1997. 751(1): p. 37-46.
23. McCarthy, K. D. and J. de Vellis, Preparation of separate astroglial and oligodendroglial cell cultures from rat cerebral tissue. J Cell Biol, 1980. 85(3): p. 890-902.
24. Barres, B. A., et al., Multiple extracellular signals are required for long-term oligodendrocyte survival. Development, 1993. 118(1): p. 283-95.
25. Yang, Z., M. Watanabe, and A. Nishiyama, Optimization of oligodendrocyte progenitor cell culture method for enhanced survival. J Neurosci Methods, 2005. 149(1): p. 50-6.
26. Hall, A. A., et al., Human umbilical cord blood cells directly suppress ischemic oligodendrocyte cell death. J Neurosci Res, 2009. 87(2): p. 333-41.
27. Okuaki, Y., et al., Splenectomy-reduced hepatic injury induced by ischemia/reperfusion in the rat. Liver, 1996. 16(3): p. 188-94.
28. Jaeschke, H., et al., Protection against TNF-induced liver parenchymal cell apoptosis during endotoxemia by a novel caspase inhibitor in mice. Toxicol Appl Pharmacol. 2000. 169(1): p. 77-83.
29. Jaeschke, H., Reactive oxygen and mechanisms of inflammatory liver injury. J Gastroenterol Hepatol, 2010. 15(7): p. 718-24.
30. Fan, C., R. M. Zwacka, and J. F. Engelhardt, Therapeutic approaches for ischemia/reperfusion injury in the liver. J Mol Med, 1999. 77(8): p. 577-92.
31. Jiang, H., et al., Splenectomy ameliorates acute multiple organ damage induced by liver warm ischemia reperfusion in rats. Surgery, 2007. 141(1): p. 32-40.
32. Savas. M. C., et al., Splenectomy attenuates intestinal ischemia-reperfusion-induced acute lung injury. J Pediatr Surg, 2003. 38(10): p. 1465-70.
33. Das et al., Lateral fluid percussion injury of the brain induces CCL20 inflammatory chemokine expression in rats. J. Neuroinflammation 2011, 8:148.
34. Ajmo et al., The spleen contributes to stroke-induced neurodegeneration, J. Neurosci. Res. 2008, 86(10): 2227-2234.

What is claimed is:

1. A method of diagnosing whether a subject has traumatic brain injury, comprising:
a) determining CC chemokine ligand 20 (CCL20) level in a biological sample obtained of the subject; and
b) comparing the CCL20 level in the biological sample of the subject to a predetermined reference value, and diagnosing the subject as having traumatic brain injury if the CCL20 level in the biological sample of the subject is higher than the predetermined reference value.

2. The method according to claim 1, wherein said method further comprises obtaining the biological sample from the subject.

3. The method according to claim 1, wherein the biological sample is selected from a blood, lymph, cerebrospinal fluid, spleen tissue, or brain tissue sample.

4. The method according to claim 1, wherein the biological sample is a blood sample.

5. The method according to claim 1, wherein the biological sample is collected within 48 hours after the primary TBI injury.

6. The method according to claim 1, wherein step (a) comprises contacting the biological sample with an agent selected from:
(1) an antibody that specifically binds to CCL20 or an antibody fragment thereof, a CCL20 binding partner, or an aptamer that specifically binds to CCL20; or
(2) an oligonucleotide complementary to a nucleic acid sequence encoding a CCL20 protein, an oligonucleotide complementary to a fragment of a nucleic acid sequence encoding a CCL20 protein, or an oligonucleotide that binds specifically to a nucleic acid sequence encoding a CCL20 protein.

7. The method according to claim 6, wherein the agent is selected from:
(1) an antibody that specifically binds to CCL20 or an antibody fragment thereof; or
(2) an oligonucleotide complementary to a nucleic acid sequence encoding a CCL20 protein, or an oligonucleotide complementary to a fragment of a nucleic acid sequence encoding a CCL20 protein.

8. The method according to claim 1, wherein the CCL20 level is determined using Western blots, Northern blots, Southern blots, enzyme-linked immunosorbent assay (ELISA), microarray, immunoprecipitation, immunofluorescence, immunocytochemistry, radioimmunoassay, polymerase chain reaction (PCR), real-time PCR, nucleic acid hybridization techniques, nucleic acid reverse transcription methods, nucleic acid amplification methods, or a combination thereof.

9. A method of diagnosing whether a subject has neurodegeneration in the brain, comprising:
a) determining CC chemokine ligand 20 (CCL20) level in the biological sample of the subject; and
b) comparing the CCL20 level in a biological sample of the subject to a predetermined reference value, and diagnosing the subject as having neurodegeneration in the brain if the CCL20 level in the biological sample of the subject is higher than the predetermined reference value.

10. The method according to claim 9, wherein said method further comprises obtaining the biological sample from the subject.

11. The method according to claim 9, wherein the biological sample is a blood sample.

12. The method according to claim 9, wherein step (b) comprises contacting the biological sample with an agent selected from:
(1) an antibody that specifically binds to CCL20 or an antibody fragment thereof; or
(2) an oligonucleotide complementary to a nucleic acid sequence encoding a CCL20 protein, or an oligonucleotide complementary to a fragment of a nucleic acid sequence encoding a CCL20 protein.

13. A method for treating a subject who has traumatic brain injury, comprising one or more of the following steps:
a) modulating or reducing CCL20 level in the subject;
b) modulating or reducing CCR6 level in the subject;
c) modulating or inhibiting binding of CCL20 to CCR6 in the subject; and
d) modulating or inhibiting CCL20 signaling in the subject.

14. The method according to claim 13, comprising administering an antibody or antibody fragment that specifically binds to CCL20 or an antibody fragment thereof, or an antibody that binds specifically to CCL20 or an antibody fragment thereof.

15. The method according to claim 12, further comprising administering to the subject an additional anti-inflammatory and/or a neuroprotective agent.

16. The method according to claim 11, wherein the method comprises reducing CCL20 expression by introducing into a cell an antisense molecule against CCL20.

17. The method according to claim 14, wherein the cell is a cell in the spleen, thymus, or the brain of the subject.

18. The method according to claim 11, wherein the method comprises reducing CCR6 expression by introducing into a cell an antisense molecule against CCR6.

19. The method according to claim 16, wherein the cell is a cell in the spleen, thymus, or the brain of the subject.

20. A method for screening for therapeutics for treatment of traumatic brain injury, comprising:
a) administering a candidate molecule to an animal subject having a traumatic brain injury,
wherein the candidate molecule is selected from an agent that modulates or reduces levels of CCL20, an agent that modulates or reduces levels of CCR6, an agent that modulates or inhibits binding of CCL20 to CCR6, an agent that modulates or inhibits CCR6 signaling, and an agent that modulates or inhibits expression of CCL20 and/or CCR6;
b) determining a level of neuroinflammation and/or neurodegeneration in brain tissue of the animal subject; and
c) selecting the candidate molecule if said molecule reduces the level of neuroinflammation and/or neurodegeneration in brain tissue of the animal subject, when compared to that of a control animal subject that received the same brain injury but is untreated with said candidate molecule.

21. A kit for diagnosis of traumatic brain injury and/or for diagnosis of neurodegeneration in the brain, wherein the kit comprises one or more of the following agents:
(1) an antibody that specifically binds to CCL20 or an antibody fragment thereof; and
(2) an oligonucleotide complementary to a nucleic acid sequence encoding a CCL20 protein, an oligonucleotide complementary to a fragment of a nucleic acid sequence encoding a CCL20 protein.

22. A therapeutic composition for treatment of traumatic brain injury and/or treatment of neurodegeneration in the brain, wherein the composition comprises one or more of the following therapeutic agents:
(1) an antibody that specifically binds to CCL20 or an antibody fragment thereof that specifically binds to CCL20, and/or an antibody that binds specifically to CCR6 or an antibody fragment thereof that specifically binds to CCR6; and
(2) an antisense molecule against CCL20, and/or an antisense molecule against CCR6.


SEQUENCE LISTING (2 sequences)

SEQ ID NO: 1 - protein, 96 aa, Homo sapiens
MCCTKSLLLAALMSVLLLHLCGESEAASNFDCCLGYTDRILHPKFIVGFTRQLANEGCDINAIIFHTKKKLSVCANPKQTWVKYIVRLLSKKVKNM

SEQ ID NO: 2 - protein, 95 aa, Homo sapiens
MCCTKSLLLAALMSVLLLHLCGESEASNFDCCLGYTDRILHPKFIVGFTRQLANEGCDINAIIFHTKKKLSVCANPKQTWVKYIVRLLSKKVKNM